(19) United States
(12) Patent Application Publication
(10) Pub. No.: US 2023/0162858 A1
(43) Pub. Date: May 25, 2023

(54) DIAGNOSTIC FOR ORAL CANCER

(71) Applicant: VIOME LIFE SCIENCES, INC., Bellevue, WA (US)

(72) Inventors: Guruduth S. BANAVAR, Pelham Manor, NY (US); Tunji OGUNDIJO, Bronx, NY (US); Hal TILY, New York, NY (US); Momchilo VUYISICH, Bothell, WA (US); Chamindie PUNYADEERA, Brisbane (AU)

(21) Appl. No.: 17/915,082

(22) PCT Filed: Mar. 28, 2021
(86) PCT No.: PCT/US21/24547

§ 371 Date: Sep. 27, 2022

Related U.S. Application Data
(60) Provisional application No. 63/001,236, filed on Mar. 27, 2020.

(51) Int. Cl. G16H 50/20; C12Q 1/6886; G16B 30/10

(52) U.S. Cl. CPC G16H 50/20; C12Q 1/6886; G16B 30/10

(57) ABSTRACT

Provided herein are systems and methods for inferring a state, e.g., presence or absence, of oral cancer in a subject. The methods involve analyzing taxa activity, microbial activity, and, optionally, host somatic cell gene activity from a sample comprising an oral microbiome of a subject, and executing a diagnostic model that infers the presence or absence of oral cancer. Further provided are methods of confirming diagnosis and for therapeutic intervention.

### REFERENCE TO RELATED APPLICATIONS

This application claims the benefit of U.S. provisional patent application 63/001,236, filed Mar. 27, 2020, the contents of which are incorporated herein in its entirety.

### STATEMENT AS TO FEDERALLY SPONSORED RESEARCH

None.

### THE NAMES OF THE PARTIES TO A JOINT RESEARCH AGREEMENT

This invention was made by or on behalf of parties to a joint research agreement entitled “Collaboration Agreement” effective as of May 13, 2019 between Viome, Inc. and Queensland University of Technology.

### SEQUENCE LISTING

None.

### BACKGROUND

Microbiome refers to the collection of microorganisms—bacteria, fungi and viruses—that inhabit the body of multicellular organisms. The microbiome inhabits many different parts of the human body, including, for example, mouth, throat, gut, skin, eye, nose, bronchi, urethra, and vagina. Microbes commonly found in the human microbiome include, for example, Escherichia, Haemophilus, Streptococcus, Neisseria, Bacteroides, Clostridium, Mycobacterium, Pseudomonas, Spirochaeta and Mycoplasma.

Microbiome composition (taxonomy) and activity can be associated with wellness and health conditions. Knowledge of such associations can be useful for the determination and treatment of such conditions. Alterations in a subject's microbiome content and activity can impact wellness and health.

Oral cancers express genes that healthy tissue does not. Oral cancer cells may also have genetic and epigenetic variations that are different from healthy tissues. These include primary sequence variants (SNPs, indels, translocations, etc.) and post-transcriptional modifications, such as RNA base modifications, splice variants, etc.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary computer system.

FIG. 2 shows the genesets with highest statistically significant overlap (FDR q-value <=0.05) in the 50 Hallmark genesets.

FIG. 3 shows the statistically significant overlap with genesets in the Catalog of Chemical and Genetic perturbations (out of 3358 genesets).

FIG. 4 shows genesets with statistically significant overlap with Canonical pathways which include 2868 genesets from KEGG, BioCarta and Reactome.

FIG. 5 shows the overlap with oncogenic signature sets.

FIG. 6 shows species features grouped by Genera and Phyla.

FIGS. 7A-7B show VFCs with both species and KOs.

### SUMMARY

In one aspect, provided herein is a method for inferring a state of oral cancer in a subject, comprising: a) providing a biological sample from a subject comprising an oral microbiome, and, optionally, somatic host cells; b) sequencing nucleic acids from the sample to produce sequence information; c) determining, from the sequence information, measures of activity of each of one or more microbial taxa and/or measures of activity of one or more gene orthologs, wherein the one or more measures are included in a feature set; d) executing by computer a classification model that infers, from one or more features in the feature set, a state of oral cancer in the subject. In one embodiment the method further comprises d) outputting the inference to a user interface device or to computer-readable memory. In another embodiment the method further comprises d) delivering and/or administering to the subject a therapeutic intervention effective to treat the oral cancer. In another embodiment the classification model classifies presence or absence of oral cancer. In another embodiment wherein the classification model classifies a stage of oral cancer (e.g., selected from stage 0, stage 1, stage 2, stage 3, stage 4). In another embodiment the nucleic acids comprise a microbial metatranscriptome. In another embodiment wherein the nucleic acids further comprise host nucleic acids. In another embodiment the subject is a human. In another embodiment the classification model uses features selected from both microbial taxa activity and gene ortholog activity. In another embodiment the classification model uses one or more features selected from the features of Table 1. In another embodiment the classification model uses at least, exactly or no more than any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, or 157 of the features selected from the features of Table 1. In another embodiment the classification model uses at least, exactly or no more than any of 1, 2, 3, 4, 5, 6, 7, 8, 9, 10, 11, 12, 13, 14, 15, 16, or 17 of the features selected from: Actinobaculum sp. oral taxon 183, Actinomyces massiliensis, Actinomyces sp. oral taxon 448, Alloscardovia omnicolens, Selenomonas sp. CM52, Mycoplasma salivarium, Parvimonas sp. oral taxon 110, Rothia sp. HMSC062H08, K01697, K12452, Actinomyces johnsonii, Prevotella loescheii, Streptococcus cristatus, Streptococcus sobrinus, Streptococcus sp. HPH0090, Tannerella forsythia, and K02909. In another embodiment the features of Table 1 include one or more microbial taxa features and/or one or more gene ortholog features. In another embodiment the features of Table 1 include one or more positively associated features and/or one or more negatively associated features. In another embodiment the classification model uses only features selected from the features of Table 1. In another embodiment the oral cancer is selected from squamous cell carcinoma, verrucous carcinoma, minor salivary gland carcinoma, lymphoma, benign oral cavity tumor and basal cell carcinoma.

In another aspect provided herein is a method comprising: a) providing biological samples from each of a first set of subjects and a second set of subjects, wherein the biological samples comprise an oral microbiome, and, optionally, somatic host cells, and wherein the first set of subjects have oral cancer present and the second set of subjects have oral cancer absent; b) sequencing nucleic acids in the biological samples to provide sequence information; and c) performing a statistical analysis on the sequence information to produce a model that infers a state of oral cancer in a subject based on sequence information. In one embodiment the statistical analysis comprises a model developed by machine learning.

In another aspect provided herein is a method comprising: a) providing a biological sample from a subject, wherein the biological sample comprises an oral microbiome; b) sequencing nucleic acids in the biological sample to provide sequence information; c) executing a model of claim 14 on the sequence information to infer a state of oral cancer in the subject based on the sequence information; and d) outputting the inference to a user interface device or to computer-readable memory.

In another aspect provided herein is a method comprising: a) administering to a subject inferred to have oral cancer by a method of claim 1 or as disclosed herein, a therapeutic intervention effective to treat the oral cancer.

In another aspect provided herein is a system comprising: (a) a computer comprising: (i) a processor; and (II) a memory, coupled to the processor, the memory storing a module comprising: (1) nucleic acid sequence information from a biological sample from a subject comprising an oral microbiome; (2) a classification model which, based on values including the measurements, classifies the subject as having oral cancer present or absent, wherein the classification model is configured to have a sensitivity of at least 75%, at least 85% or at least 95%; and (3) computer executable instructions for implementing the classification model on the test data.

In another aspect provided herein is a method for developing a computer model for inferring, from feature data, a state of oral cancer in a subject, the method comprising: a) training a machine learning algorithm on a training data set, wherein the training data set comprises, for each of a plurality of subjects, (1) a class label classifying a subject as having or not having an oral cancer; and (2) feature data comprising quantitative measures for each of a plurality of features selected from oral microbiome transcriptome expression, and wherein the machine learning algorithm develops a model that infers a class label for a subject based on the feature data.

In another aspect provided herein is a method that infers a state of oral cancer in a subject, the method comprising: (a) providing a data set comprising, for the subject, feature data for each of a plurality of features selected from oral microbiome transcriptome gene expression data and taxa activity data; and (b) executing a computer model on the data set to infer the presence or absence of oral cancer in the subject.

In another aspect provided herein is a software product comprising a computer readable medium in tangible form comprising machine executable code, which, when executed by a computer processor, infers a state of oral cancer in a subject by: (a) accessing a data set comprising, for a subject, feature data for each of a plurality of features selected from oral microbiome transcriptome gene expression data and taxa activity data; and (b) executing a computer model on the data set to infer the state of oral cancer in the subject.

In another aspect provided herein is a method of treating oral cancer in a subject comprising: (a) determining the presence of oral cancer in a subject according to a method as described herein; and (b) administering a therapeutic intervention to the subject effective to treat the oral cancer.

In another aspect provided herein is a method for diagnosing and treating an oral cancer in a subject, the method comprising: (a) receiving from a subject a sample comprising an oral microbiome and, optionally, host somatic cells; (b) determining nucleic acid sequences of a microorganism component of the sample; (c) determining alignments of the nucleic acid sequence to reference nucleic acid sequences associated with the oral cancer; (d) generating a microbiome feature dataset for the subject based upon the alignments; (e) generating an inference of the oral cancer in the subject upon processing the microbiome feature dataset with an inference model derived from a population of subjects; and (f) at an output device associated with the subject, providing a therapy to the subject with the oral cancer upon processing the inference with a therapy model designed to treat the oral cancer.

In another aspect provided herein is a method comprising: (a) measuring, in a sample from a subject comprising an oral microbiome and, optionally, host somatic cells, activity of one or more biomarkers selected from Table 1; (b) inferring, from the measurements, presence of oral cancer in the subject; and (c) delivering to the subject a therapeutic intervention to treat the oral cancer. In one embodiment measuring comprises: (i) optionally, amplifying microbial metatranscriptome sequences in the sample; (ii) sequencing the microbial metatranscriptome from the sample to produce sequence reads; (iii) searching reference sequences in a reference sequence catalog for matches with the sequence reads; (iv) determining amounts of sequence reads matching references sequences in the catalog to produce a data set; and (v) determining, from the data set, activity of each of the one or more biomarkers. In another embodiment determining activity comprises: (1) for biomarkers that are taxa categories, performing a taxonomic analysis with a metagenomic classifier to measure taxa activity; (2) for biomarkers that are gene orthologs, performing a functional analysis by determining activity of genes having the same function across taxa based on sequences corresponding to microbial open reading frames (ORFs), and combing the activities to produce gene ortholog activity. In another embodiment inferring comprises: (i) executing by computer a classification model that infers presence or absence of oral cancer based on the biomarkers. In another embodiment the therapeutic intervention is selected from a drug, a dietary supplement, a food ingredient, and a food. In another embodiment measuring comprises: (i) selectively amplifying in the sample nucleic acids specific for the biomarkers; and (ii) determining amounts of the amplified nucleic acids.

In another aspect provided herein is a method comprising: a) providing biological samples from each of a first set of subjects and a second set of subjects having an oral cancer and having been subject to a therapeutic intervention, wherein the biological samples comprise an oral microbiome, and, optionally, host somatic cells, and wherein the first set of subjects responded positively to the therapeutic intervention and the second set of subjects did not respond positively to the therapeutic intervention; b) sequencing nucleic acids in the biological samples to provide sequence information; and c) performing a statistical analysis on the sequence information to produce a model that infers subject oral cancer having a positive response or lack of positive response to the therapeutic intervention.

In another aspect provided herein is a method of treating a subject with oral cancer comprising: (a) inferring that the subject will respond positively to each of one or more therapeutic interventions by executing a model on nucleic acid information from a biological sample from the subject comprising or oral microbiome and, optionally, host somatic cells; and (b) administering to the subject one or more of the therapeutic interventions.

### DETAILED DESCRIPTION

### I. Introduction

Oral cancers will interact with the oral microbiome such that the microbes express genes, resulting in transcripts, that may not be expressed in the absence of oral cancers. Such transcripts may be found in saliva and be identified as biomarkers of oral cancer. By analyzing oral metatranscriptome, biomarkers of oral cancers may be found in the combination of human and microbial transcripts found in the mouth.

It has been discovered that features of a subject's oral metatranscriptome (RNA content) are associated with oral cancer. Accordingly, disclosed herein are methods for analyzing the oral metatranscriptome (MT), producing oral MT data, building machine-learning models to learn associations between oral cancers and MT data, and the use of such models to determine the presence or absence of oral cancer in a subject, as well as methods of treatment following such determination.

Methods of diagnosing oral cancer use a mouth sample from a subject. RNA from the mouth sample is sequenced to produce nucleic acid sequence information. For gene expression analysis only, an alternative method, such as microarray, could be used. RNA sequence information is subject to bioinformatics processing. Bioinformatics processing can produce information that indicates a measure of each of a plurality of genes or gene orthologs and of active microbial taxa in the sample. It can also produce information about the sequence and level of expression of human genes and transcripts, including specific sequence variants. These data, in turn, can be used as features in a dataset used to perform statistical analysis, e.g., to train a machine learning algorithm, to develop a model to classify a sample as consistent with presence of oral cancer or absence of oral cancer, or with a probability of cancer. Such models can be implemented on samples from test subjects. Subjects diagnosed with oral cancer according to the methods described herein can be administered a therapeutic intervention to treat the cancer.

### I. Sample Collection and Processing

### A. Subjects

The term “subject” refers to any animal. Animals can include vertebrates or invertebrates, including fish, amphibians, reptiles, birds and mammals. Mammalian hosts can include primates and, in particular, humans. Mammalian subjects also can include farm animals and companion animals. The term “host” refers to a subject organism serving a vehicle for habitation of a microbiome. Because certain methods described herein include sequencing of a subject's microbiome, such subjects may also be referred to as “hosts.”

A human subject can be more than 20 years old or more than 50 years old. A subject can have a history of tobacco use or no history of tobacco use. As used herein, a subject with a history of tobacco use can be a current tobacco user or a former tobacco user. A current tobacco user is one who uses tobacco products four or more times per week in the past six months. A former tobacco user is one who has quit using tobacco products at the current time, but had previously used tobacco products four or more times per week for six months or more, within the last 20 years. A subject with no history of tobacco use is neither a current tobacco user of a subject with a history or tobacco use, that is, not being a tobacco user for at least twenty years.

### B. Biological Samples

As used herein, the term “microbiome” includes a microbial community comprising one or a plurality of different microbial taxa inhabiting a host. As used herein, the term “oral microbiome” refers to a microbiome inhabiting a mouth (e.g., tongue, gums, cheek, saliva) or throat, of a host.

As used herein, the term metatranscriptome (MT) refers to the collection of microbial and, optionally, host, transcripts in a sample. Accordingly, a mouth metatranscriptome includes all microbiome and, optionally, host, components. Host components include any transcripts from somatic cells of the host and, in the case of an oral sample, in the mouth.

As used herein, the term “biological sample” refers to a sample that includes material of biological origin, such as cells, biological macromolecules (e.g., nucleic acids, proteins, carbohydrates or lipids) or their derivatives. Saliva is an exemplary biological sample.

As used herein, the term “mouth-sourced cell” refers to a cell sourced from the mouth of a subject. This includes, without limitation, cells from the mouth microbiome and host somatic cells, such as cheek cells, tongue cells, gum cells, etc.

Samples for diagnosis of oral cancer can comprise biological samples comprising a mouth MT of a subject. Mouth MT samples can be collected, for example, from saliva, sputum or a cheek swab from a subject.

Data used in developing a model to make the inferences described herein typically comprise large data sets including thousands, tens of thousands, hundreds of thousands or millions of individual measurements taken from or about a subject, typically at the systems biology level. The data can be derived from one or more (typically a plurality) different biological system components. These biological system components, also referred to herein as “feature groups”, include, without limitation, the genome (genomic), the epigenome (epigenomic), the transcriptome (transcriptomic), the proteome (proteomic), the metabolome (metabolomic), the organismal cellular lipid components (lipidome), organismal sugar components of complex carbohydrates (glycomic), the proteome and/or genome of the immune system (immunomics) component of a system, organism phenotype (phenome, phenomic, phenotypic) and environmental exposure (exposome). (These are generally referred to herein as “-omic” data or information.)

A mouth MT sample can be preserved for transport to a laboratory. The sample can be deposited into a container that comprises an aqueous liquid, e.g., a buffered solution. The aqueous liquid can further contain reagents to inhibit or slow degradation of one or more kinds of nucleic acid, such as DNA or RNA. As used herein, the term “nucleic acid preservative” refers to a compound or composition that inhibits degradation of nucleic acid. RNA preservatives include, without limitation, formalin, sulfate (e.g., ammonium sulfate), isothiocyanate (e.g., guanidinium isothiocyanate) and urea. Commercially available RNA preservatives include, for example, TRIzol (ThermoFisher), RNAlater (Ambion, Austin, Tex., USA), Allprotect tissue reagent (Qiagen), PAXgene Blood RNA System (PreAnalytiX GmbH, Hombrechtikon), RNA/DNA Shield® (Zymo Research, Irvine, Calif.), and DNAstable (MilliporeSigma, Burlington, Mass.).

### C. Sample Processing

Sample processing can proceed with cell lysis. Cell lysis can be performed by any method known in the art this can include, for example, bead beading, a method that involves rapidly shaking a container containing solid particles such that cells in the container are lysed.

Polynucleotides can be extracted directly from the sample, or cells in the sample can first be lysed to release their polynucleotides. In one method, lysing cells comprises bead beating (e.g., with zirconium beads). In another method, ultrasonic lysis is used. Such a step may not be necessary for isolating cell-free nucleic acids.

After cell lysis, samples are further processed by the extraction or isolation of biomolecules in the container, e.g., biomolecules released from lysed cells. Isolated biomolecules typically include nucleic acids such as DNA and/or RNA. Other biomolecules to be isolated can include polypeptides, such as proteins.

Isolation of biomolecules can be performed with a liquid-handling robot. After cell lysis, biological molecules, such as nucleic acids can be isolated or extracted from the sample

Nucleic acids can be isolated from the sample by any means known in the art. Polynucleotides can be isolated from a sample by contacting the sample with a solid support comprising moieties that bind nucleic acids, e.g., a silica surface. For example, the solid support can be a column comprising silica or can comprise paramagnetic carboxylate coated beads or a silica membrane. After capturing nucleic acids in a sample, the beads can be immobilized with a magnet and impurities removed. In another method, nucleic acids can be isolated using cellulose, polyethylene glycol, or phenol/chloroform.

If the target polynucleotide is RNA, the sample can be exposed to an agent that degrades DNA, for example, a DNase. Commercially available DNase preparations include, for example, DNase I (Sigma-Aldrich), Turbo DNA-free (ThermoFisher) or RNase-Free DNase (Qiagen). Also, a Qiagen RNeasy kit can be used to purify RNA.

In another embodiment, a sample comprising DNA and RNA can be exposed to a low pH, for example, pH below pH 5, below pH 4 or below pH 3. At such pH, DNA is more subject to degradation than RNA.

DNA can be isolated with silica, cellulose, or other types of surfaces, e.g., Ampure SPRI beads. Kits for such procedures are commercially available from, e.g., Promega (Madison, Wis.) or Qiagen (Venlo, Netherlands).

Isolation of nucleic acids can further include elimination of non-informative RNA species from the sample. As used herein, the term “non-informative RNA” refers to a form of non-target or non-analyte species of RNA. Non-informative RNA species can include one or more of: human ribosomal RNA (rRNA), human transfer RNA (tRNA), microbial rRNA, and microbial tRNA. Non-informative RNA species can further comprise one or more of the most abundant mRNA species in a sample, for example, hemoglobin and myoglobin in a blood sample. Non-informative RNAs can be removed by contacting the sample with polynucleotide probes that hybridize with the non-informative species and that are attached to solid particles which can be removed from the sample. Examples of sequences that can be removed include microbial ribosomal RNA, including 16S rRNA, 5S rRNA, and 23S rRNA. Other examples of sequences that can be removed include host RNA. Examples include host rRNA, such as 18S rRNA, 5S rRNA, and 28S rRNA.

Isolated nucleic acids can be further processed to produce nucleic acid libraries. Production of nucleic acid libraries typically includes, in the case of RNA, converting RNA into DNA, e.g., by reverse transcription. Adaptors adapted for the DNA sequencing instrument to be used are typically attached to the DNA molecules.

According to one method, RNA molecules are reverse transcribed into cDNA using a reverse transcriptase. In certain embodiments, primers comprising a degenerate hexamer at their 3′ end hybridize to RNA molecules. The reverse transcriptase extends the primer and can leave a terminal poly-G overhang. In certain embodiments, the primer can also comprise adapter sequences. A template molecule comprising a Poly-C overhang and, optionally, adapter sequences, can be hybridized to the poly-G overhang and used to guide extension to produce an adapter tagged cDNA molecule comprising a cDNA insert flanked by adapter sequences.

If the target polynucleotide is DNA, then DNA can be isolated with silica, cellulose, or other types of surfaces, e.g., Ampure SPRI beads. Kits for such procedures are commercially available from, e.g., Promega (Madison, Wis.) or Qiagen (Venlo, Netherlands).

Methods of enriching nucleic acid samples include the use of oligonucleotide probes. Such probes can be used for either positive selection or negative selection. Such methods often reduce the amount of non-target nucleotides.

Adapter tagged cDNA molecules can be amplified using well-known techniques such as PCR, to produce a library.

In certain embodiments the nucleic acids to be sequenced are comprised in the transcriptome. As used herein, the term “metatranscriptome” refers to the set of RNA molecules in a population of cells. This can include all RNAs, but sometimes refers to only mRNA. In the present context it generally refers to RNA molecules produced by either human or microbial cells. In certain embodiments, the nucleic acids to be sequenced can be free or essentially free of host nucleic acids (“host-free nucleic acids”).

### D. Nucleic Acid Sequencing

The isolated nucleic acids are generally sequenced for subsequent analysis. The methods described herein generally employ high throughput sequencing methods. As used herein, the term “high throughput sequencing” refers to the simultaneous or near simultaneous sequencing of thousands of nucleic acid molecules. High throughput sequencing is sometimes referred to as “next generation sequencing” or “massively parallel sequencing.” Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing (Complete Genomics), Heliscope single molecule sequencing, single molecule real time (SMRT) sequencing (PacBio), and nanopore DNA sequencing (e.g., Oxford Nanopore). Nucleotide sequences of nucleic acids produced by sequencing are referred to herein as “sequence information” or “sequence data”.

Also provided herein are methods of analyzing RNA transcripts in a heterogeneous microbial sample. The RNA transcripts can be part of a transcriptome for a cell or cells in the heterogeneous microbial sample. Information regarding the transcriptomes of a plurality of cells from different species may be obtained. The methods generally include isolating and sequencing the RNA found in a sample as described above.

### E. Bioinformatics

The sequences obtained from these methods can be preprocessed prior to analysis. If the methods include sequencing a transcriptome, the transcriptome can be preprocessed prior to analysis. In one method, sequence reads for which there is paired end sequence data are selected. Alternatively, or in addition, sequence reads that align to a reference genome of the host are removed from the collection. This produces a set of host-free transcriptome sequences. Alternatively, or in addition, sequence reads that encode non-target nucleotides can be removed prior to analysis. As described above, non-target nucleotides include those that are over-represented in a sample or non-informative of taxonomic information. Removing sequence reads that encode such non-target nucleotides can improve performance of the systems, methods, and databases described herein by limiting the sequence signature database to open reading frames (a part of a reading frame that has the ability to be translated) can the size of the database, the amount of memory required to run the sequence signature generation analysis, the number of CPU cycles required to run the sequence signature generation analysis, the amount of storage required to store the database, the amount of time needed to compare sample sequences to the database, the number of alignments that must be performed to identify sequence signatures in a sample, the amount of memory required to run the sequence signature sample analysis, the number of CPU cycles required to run the sequence signature sample analysis, etc.

### 1. Taxonomic Data

Subject data can include taxonomic data about the taxonomic classification and amounts of microbes in a microbiome of the subject. Such data is typically derived from nucleic acid sequence data obtained from the subject's microbiome. 16S RNA sequences are a standard source of information for assigning taxonomic classifications. Non-rRNA transcriptome data as an alternative source of information for taxonomic classification. Such methods are described in international patent publication WO 2018/160899 (“Systems And Methods For Metagenomic Analysis”). Many metagenomic classifiers, aligners and profilers are publicly available. See, for example, Florian P Breitwieser et al., “A review of methods and databases for metagenomic classification and assembly,” Briefings in Bioinformatics, Volume 20, Issue 4, July 2019, Pages 1125-1136, doi.org/10.1093/bib/bbx120, Published: 23 Sep. 2017. These include, without limitation, Centrifuge, GOTTCHA, kraken, kraken2, CLARK, Kaiju, MetaPhlAn, MetaPhlAn2, MEGAN, LMAT, MetaFlow, mOTUs, and mOTUs2.

Another method of analysis includes analysis of composition of microbiomes (“ANCOM”). This method is described in, for example, Mandel S, et al., “Analysis of composition of microbiomes: a novel method for studying microbial composition”, Microb Ecol Health Dis. 2015 May 29; 26:27663. doi: 10.3402/mehd.v26.27663. eCollection 2015.

Taxonomic analysis can involve searching a sequence catalog of microbiome sequences for matches with sequences in the dataset, e.g., metatranscriptomic sequences. Matches are assigned to the proper taxonomic category. Numbers of matches with a taxonomic category can indicate quantities of microbes of that taxonomic category in the sample.

The classifications can be at one or a plurality of different taxonomic levels, typically down to the species or strain level. Sequencing reads that map to sequences in the sub-catalog can then be labeled with tags indicating the taxonomic category at each level. The taxonomic label is assigned. Such systems can include classical or modern taxonomic classification systems.

As used herein, the term “taxon” (plural “taxa”) is a group of one or more populations of an organism or organisms seen by taxonomists to form a unit. A taxon is usually known by a particular name and given a particular ranking. For example, species are often designated using binomial nomenclature comprising a combination of a generic name for the genus and a specific name for the species. Likewise, subspecies are often designated using trinomial nomenclature comprising a generic name, a specific name, and a subspecific name. The taxonomic name for an organism at the taxonomic rank of genus is the generic name, the taxonomic name for an organism at the taxonomic rank of species is the specific name, and the taxonomic name for an organism at the taxonomic rank of subspecies is the subspecific name, when appropriate.

As used herein, the term “taxonomic level” refers to a level in a taxonomic hierarchy of organisms such as, strain, species, genus, family, order, class, phylum, and kingdom. In some embodiments, each taxonomic level includes a plurality of “taxonomic categories”, that is, the different categories belonging to particular taxonomic level. Some taxonomic levels only include a single member.

As used herein, the term “species” is intended to encompass both morphological and molecular methods of categorization. Species can be defined by genetic similarity. In some embodiments, a cladistic species is an evolutionarily divergent lineage and is the smallest group of populations that can be distinguished by a unique set of morphological or genetic traits.

Genomes imported into the reference catalog are typically indexed with a genome number. Various taxonomy indices, such as the NCBI taxonomy, categorized each genome number into a taxonomic classification. Consequently, sequencing reads that match reference sequences can also be taxonomically classified based on the number. Accordingly, using a taxonomic tree implicit in the taxonomic designation taxonomic source of any sequencing read can be identified and classified.

Once classified, sequences in each category can be quantified or estimated to determine amounts of sequencing reads in each taxonomic category and the relative abundance of each taxonomic entity. The sequencing reads can be metatranscriptomic in origin. Accordingly, amounts of reads in a taxon represent transcriptional activity of the taxon, rather than pure numbers of organisms in the taxon in the sample. “Activity of a microbial taxon” can refer to transcriptional activity.

### 2. Gene Expression Quantification

The methods, systems and databases herein can be used to identify activity of a gene, a biochemical pathway or a functional activity from microbes present in the sample. In some embodiments, the methods include aligning sequencing reads to a database comprising open reading frame information that is associated with a particular biochemical activity or pathway. Some of such methods can include identifying taxonomic information for a sequence. Examples include the VIOMEGA algorithm (see WO 2018/160899 (Vuyisich et al.) or GOTTCHA algorithm, which detects sequence signatures that identify nucleic acids as originating from organisms at various taxonomic levels. Nucleic Acids Res. 2015 May 26; 43(10): e69. Other methods include MetaPhlAn, Bowtie2, mOTUs, Kraken, and BLAST. Some of such methods do not include identifying taxonomic information for the sequence, but instead may identify the biochemical activity, pathway, protein, functional RNA, product, or metabolite associated with a particular sequence read or sequence signature.

“Gene expression,” “gene activity” or “activity of a gene” is generally a function of transcription, e.g., the quantity of RNA in a sample encoding the gene. This can be done at any taxonomic level. For example, gene activity could be a measure of activity of the gene in a single species, or it could be activity of the gene across organisms belonging to a common genus, class, order or phylum. Thus, the term “gene” can refer to orthologs of a gene across different species. As used herein, the term “gene ortholog” refers to a homologous version of a gene across different taxa having the same biological function. Typically, gene orthologs share a high degree of sequence identity. Such orthologs can be identified, for example, with the KEGG orthology. Kanehisa, M. and Goto, S.; KEGG: Kyoto Encyclopedia of Genes and Genomes. Nucleic Acids Res. 28, 27-30 (2000)). KO (KEGG Orthology) databases. The KO (KEGG Orthology) database is a database of molecular functions represented in terms of functional orthologs. The KO databases include, among other things, genomic information, chemical information and systems information such as biological pathway maps. A functional ortholog is manually defined in the context of KEGG molecular networks, namely, KEGG pathway maps, BRITE hierarchies and KEGG modules. In the KEGG orthology, orthologs are identified by number. So, for example, “K01808” refers to rpiB, ribose 5-phosphate isomerase B [EC:5.3.1.6]. Search at the world wide web site genome.jp/kegg/kegg2.html.

Nucleic acid sequence information is processed using bioinformatics to extract higher order information. In particular, two types of information that are usefully extracted from sequence data include gene activity information and taxa activity information.

The activities of one or more taxa groups can be determined from the amount of nucleic acid, e.g., RNA, in a sample originating from particular taxonomic groups. Microbial taxa include taxonomic designation at any taxonomic level, e.g., species, genus, order, class, or phylum. Active microbial taxa are taxa that are not merely present but that are metabolically active, e.g., as measured by transcriptional levels of the microbial genome. Taxa groups of interest include, without limitation, Prevotella (genus)/Bacteroides (genus) ratio, Eubacterium rectale (species), Eubacterium eligens (species), Faecalibacterium prausnitzii (species), Akkermansia muciniphila (species), metabolic-related probiotic species (functional group), Roseburia (genus), Bifidobacterium (genus), Lactobacillus (genus), Clostridium butyricum (species), Allobaculum (genus), Firmicutes (phylum)/Bacteroidetes (phylum) ratio, Lachnospiraceae (family), Enterobacteriaceae (family), Ralstonia pickettii (species), Bilophila wadsworthia (species).

Similar bioinformatic approaches can be used to analyze human gene expression, by identifying and counting the transcripts produced by human cells. Bioinformatic software to extract such information from sequence data is known in the art. Examples include the VIOMEGA algorithm (see WO 2018/160899 (Vuyisich et al.) or GOTTCHA algorithm, which detects sequence signatures that identify nucleic acids as originating from organisms at various taxonomic levels. Nucleic Acids Res. 2015 May 26; 43(10): e69. Other methods include MetaPhlAn, Bowtie2, mOTUs, Kraken, BLAST and Salmon.

“Functional activities” are biological activity categories including biological or health functions or conditions at the cellular, organ or organismal level. Functional activities are assigned functional activity scores based on such data. Functional activity scores represent quantitative measures of functional activity. A functional category can involve any function related to health or wellness. Functional categories can embrace health parameters, health indicators, biological conditions and health risks. The activity of the function is assessed by analyzing -omic, e.g., transcriptomic data, which is collected from active, living organisms, e.g., expressing RNA from their genomes.

Functional activity includes integrative functional activities and non-integrative functional activities. Non-integrative functional activities are based on a single type of data or function, such as microbiome pathway activity data, taxa group activity data and host transcriptomic data. Integrative functional activities can be based on a plurality of different kinds of data or functions. For example, such functional activities can combine pathway activity data in taxa activity data.

In certain embodiments, functional activities include the activities of one or more pathways. As used herein, the term “pathways” refers to biological pathways, which are sequences of proven molecular events (such as enzymatic reactions or signal transduction or transport of substances or morphological structure changes) that lead to specific functional outcomes (such as secretion of substances, sporulation, biofilm formation, motility). Many biological pathways are known in the art, and examples can be found on the web at wikipathways.org/index.php/WikiPathways, pathwaycommons.org, and proteinlounge.com/Pathway/Pathways.aspx. Manual expert curation of scientific literature also can be used to reconstruct or create custom biological pathways. Biological pathways can include a number of genes that encode peptides or proteins, which play specific signaling, metabolic, structural or other biochemical roles in order to carry out various molecular pathways.

As used herein, the terms “biochemical activity” and “biochemical pathway activity” refer to activity of a biochemical pathway. Pathways of interest include, without limitation, butyrate production pathways, LPS biosynthesis pathways, methane gas production pathways, sulfide gas production pathways, flagellar assembly pathways, ammonia production pathways, putrescine production pathways, oxalate metabolism pathways, uric acid production pathways, salt stress pathways, biofilm chemotaxis in virulence pathways, TMA production pathways, primary bile acid pathways, secondary bile acid pathways, acetate pathways, propionate pathways, branched chain amino acid pathways, long chain fatty acid metabolism pathways, long chain carbohydrate metabolic pathways, cadaverine production pathways, tryptophan pathways, starch metabolism pathways, fucose metabolism pathways.

### II. Data Collection

In order to build models to make inferences about the presence or absence of oral cancer, a dataset must be assembled that includes data from a plurality of subjects. Subjects typically will include both those diagnosed as having oral cancer and those diagnosed as not having oral cancer. The number of subjects in each category should be sufficient to provide statistically meaningful results. For example, such a cohort can comprise at least any of 50, 100, 500, or 1000 subjects diagnosed with the disease and at least any of 50, 100, 500, or 1000 subjects diagnosed without the disease.

### III. Statistical Analysis

A. Data sets

In building or executing a model to predict the oral cancer of an individual subject, databases are provided that include information about one or a plurality of subjects. Raw data can include sequence data or information derived therefrom.

Models, or classification models, are algorithms that make inferences based on feature data measured from a test. Methods of generating models to predict oral cancer can involve providing a training dataset on which a machine learning algorithm can be trained to develop one or more models to predict oral cancer. The training dataset will include a plurality of training examples or instances, typically for each of a plurality of subjects and typically in the form of a vector. Each training example will include a plurality of features and, for each feature, data, e.g., in the form of numbers or descriptors. Where learning is to be supervised, the data will include a classification of the subject into a category of a categorical variable to be inferred. For example, the categorical variable may be “cancer diagnosis” and the categories or classifications of this variable can be “present” and “absent”. Typically, for machine learning, the training examples will have at least 10, at least 100, at least 500 or at least 1000 different features. The features selected are those on which prediction will be based. In the present case features can include genes or taxa or gene activity and/or taxa activity. The collection of features included in a dataset can be referred to as a “feature set”.

Accordingly, the collection of sequence data or gene activity and/or taxa activity data from an individual subject represent data for a particular instance. Each gene or taxon measured or determined represents a feature. A value, which can be a number or qualifier, is provided for an instance at a particular feature. The collection of data across a plurality of instances or examples, e.g. subjects, represents a dataset. Accordingly, each dataset can be represented as a vector of values for combinations of instances and features.

A measurement of a variable, such as a phenotypic trait (e.g., presence or absence of cancer), quantity of microbes in a taxon, gene expression levels, biochemical pathway activity or a functional activity, can be any combination of numbers and words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category (e.g., a class label), such a “cancer” or “non-cancer”, “old” or “young”, “form 1” or “form 2”, “subject 1 . . . subject n,” etc. Measurements of a variable on an ordinal scale produce a ranking, such as “first”, “second”, “third”; or order from most to least. Measurements on a ratio scale include, for example, any measure on a pre-defined scale, such as number of molecules, weight, activity level, signal strength, concentration, age, etc., as well as statistical measurements such as frequency, mean, median, standard deviation, or quantile. Measurements on a ratio scale can be relative amounts or normalized measures. Quantitative measures can be given as a discrete or continuous range. Examples of quantitative measures include a number, a degree, a level, a range or bucket. A number can be a number on a scale, for example 1-10. Alternatively, the score can embrace a range. For example, ranges can be high, medium and low; severe, moderate and mild; or actionable and non-actionable. Buckets can comprise discrete numerals, such as 1-3, 4-6 and 7-10.

### B. Model Generation and Predicting Oral Cancer

Models can be created by statistical methods. Statistical analysis can include any useful methodology including, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test). Statistical analysis can be performed by hand or by computer. Computer methods include, for example, machine learning algorithms.

Machine learning involves training machine learning algorithms on training data sets comprising data from a plurality of test subjects. Machine learning algorithms are trained on the training dataset to generate models that predict the oral cancer of an individual based on sequence data or information derived therefrom. Predicted oral cancer can be translated into recommendations to the subject about therapeutic interventions to be taken.

The machine learning algorithm can be any suitable supervised machine learning algorithm, parametric or non-parametric. Machine learning algorithms include, without limitation, artificial neural networks (e.g., back propagation networks), decision trees (e.g., recursive partitioning processes, CART), random forests, discriminant analyses (e.g., Bayesian classifier or Fischer analysis), linear classifiers (e.g., multiple linear regression (MLR), partial least squares (PLS) regression, principal components regression (PCR)), mixed or random-effects models, non-parametric classifiers (e.g., k-nearest neighbors), support vector machines, and ensemble methods (e.g., bagging, boosting).

Methods for generating models to predict oral cancer can comprise the following operations. A dataset as described above is provided. The dataset includes, for each of a plurality of subjects, raw or processed data. The data set is used as a training dataset to train a machine learning algorithm to produce one or more models that predict oral cancer of a subject based on biomarkers identified from the data.

Biomarkers can be individual features used by the model in making an inference (e.g., diagnosis) of the category in question. For example, of thousands of features used in the original training dataset, the model may use no more than any of 1, 5, 10, 50, 100 or 500 features in determining the classification.

### C. Validation

A model may be subsequently validated using a validation dataset. Validation datasets typically include data on the same features as the training dataset. The model is executed on the training dataset and the number of true positives, true negatives, false positives and false negatives is determined, as a measure of performance of the model.

The model can then be tested on a validation dataset to determine its usefulness. Typically, a learning algorithm will generate a plurality of models. In certain embodiments, models can be validated based on fidelity to standard clinical measures used to diagnose the condition under consideration. One or more of these can be selected based on its performance characteristics.

### IV. Inferring Oral Cancer in a Subject

Inferring a state of oral cancer in subject generally means using a model to assign a class label related to oral cancer to a test subject. The classifier can classify the condition according to any classification scheme useful to the operator. The class label can be “presence of oral cancer” or “absence of oral cancer”, or “likely presence of oral cancer” or “likely absence of oral cancer”. Alternatively, the class label can be a stage of oral cancer, including absence of oral cancer. Alternatively, the class label can be a type of oral cancer present, or the absence of oral cancer.

Oral cancers, the presence or absence of which can be inferred by the methods described herein include, without limitation, cancer of the lip, tongue, inner lining of the cheek, gums, floor of the mouth and hard and soft palate. They further include

Methods described herein can infer a stage of an oral cancer. Oral cancer stages include the following: squamous cell carcinoma, verrucous carcinoma, minor salivary gland carcinoma, lymphoma, benign oral cavity tumors and basal cell carcinomas.

Stage 0 oral cancer: Cancer limited to layer of cells lining the oral cavity or oropharynx (also referred to as “carcinoma in situ”. Treatment may include surgery, radiation, or a combination of both.

Stage 1 oral cancer: Tumor is 2 centimeters (cm) (about ¾ inches) or less in size. The cancer has not spread to the lymph nodes or to other places in the body. Also classified as “T1, N0, and M0” where T refers to tumor size, N refers to involvement of lymph nodes, and M refers to metastasis. Treatment may include surgery, radiation, or a combination of both.

Stage 2 oral cancer: Tumor is between 2 and 4 cm (about 1½ inches) in size. The cancer has not spread to the lymph nodes or other places in the body. Also classified as T2, N0, and M0. Treatment may include surgery, radiation, or a combination of both.

Stage 3 oral cancer: Tumor is larger than 4 cm (about 2 inches) and has not metastasized, but may have spread to the lymph nodes. Also classified as T3, N0, M0; T1, N1, M0; T2, N1, M0; and T3, N1, M0. Surgery or radiation or both are likely treatment options. Chemotherapy may be suggested to destroy any cancer that has spread, and other options include targeted treatments which target specific cancer cells in oral cancer called epidermal growth factor receptor (EGFR). The drug cetuximab specifically targets EGFR cells.

Stage 4 oral cancer: Tumor can be any size, but the cancer has spread to the lymph nodes or other parts of the body. Also classified as T(1 to 4), N number (0 to 3), and either M0 or M1. Treatment may include surgery, radiation, chemotherapy, targeted treatments, or a combination.

The model selected can either result from operator executed statistical analysis or machine learning. In any case, the model can be used to make inferences (e.g., predictions) about a test subject. Test data can be generated from a sample taken from the test subject. The test dataset can include all of the same features used in the training dataset, or a subset of these features. Such a subset function as biomarkers. The model is then applied to or executed on the test dataset. Inferring oral cancer is a form of executing a model. The inference is typically performed by computer, but can be performed by a person. The choice may depend on the complexity of the operation of correlating. This produces an inference, e.g., a classification of a subject as belonging to a class (such as a diagnosis of oral cancer).

The classifier or model may generate, from the subject data, a single diagnostic number which functions as the model. Classifying a subject as having oral cancer can involve determining whether the diagnostic number is above or below a threshold (“diagnostic level”). The threshold can be determined, for example, based on a certain deviation of the diagnostic number above subject who do not have oral cancer. A measure of central tendency, such as mean, median or mode, of diagnostic numbers can be determined in a statistically significant number of normal and abnormal individuals. A cutoff above normal amounts can be selected as a diagnostic level of oral cancer. That number can be, for example, a certain degree of deviation from the measure of central tendency, such as variance or standard deviation. In one embodiment the measure of deviation is a Z score or number of standard deviations from the normal average.

The model used to make an inference of oral cancer can be chosen to have any desired level of sensitivity, specificity positive predictive value or negative predictive value.

Sensitivity refers to a value calculated according to the formula TP/(TP+FN), where TP is the number of true positive measurements (e.g., correctly inferring the presence of oral cancer in a subject) and FN is the number of false negative measurements (e.g., incorrectly inferring the absence of oral cancer in a subject). Sensitivity measures the percentage of subjects that actually have oral cancer who are inferred to have oral cancer by the test. In some embodiments, the diagnostic test can infer a presence or an absence of oral cancer with a sensitivity of greater than about any of: 70%, 75%, 80%, 85%, 86%, 87%, 88%, 89%, 90%, 91%, 92%, 93%, 94%, 95%, 96%, 97%, 98%, 99%, or 99.5%.

Specificity refers to a value calculated according to the formula TN/(TN+FP), where TN is the number of true negative measurements (e.g., correctly inferring an absence of oral cancer in a subject) and FP is the number of false positive measurements (e.g., incorrectly inferring the presence of oral cancer in a subject). Specificity measures the percentage of subjects that actually do not have oral cancer who are inferred to not have oral cancer by the test. In some embodiments, the diagnostic test can infer a presence or an absence of oral cancer with a specificity of greater than about any of: 70%, 75%, 80%, 85%, 86%, 87%, 88%, 89%, 90%, 91%, 92%1, 93%, 94%, 95%, 96%, 97%, 98%, 99%, or 99.5%.

Positive Predictive Value (PPV) refers to a value calculated according to the formula TP/(TP+FP). A PPV value is the proportion of subjects inferred to be positive (presence of oral cancer) that actually have oral cancer. In some embodiments, the model, e.g., diagnostic test, may infer a presence or an absence of oral cancer in a subject at a PPV of greater than about any of: 70%, 75%, 80%, 85%, 86%, 87%, 88%, 89%, 90%, 91%, 92%, 93%, 94%, 95%, 96%, 97%, 98%, 99%, or 99.5%.

Negative Predictive Value (NPV) refers to a value calculated according to the formula TN/(TN+FN). An NPV value is the proportion of subjects inferred to be negative (absence of oral cancer) that actually do not have oral cancer. In some embodiments, the model, e.g., diagnostic test, may infer a presence or an absence of oral cancer in a subject an NPV of greater than about any of: 70%, 75%, 80%, 85%, 86%, 87%, 88%, 89%, 90%, 91%%, 92%, 93%, 94%, 95%, 96%, 97%, 98%, 99%, or

Accuracy can be measured by the percentage of subjects who test positive or negative that are true positives or true negatives, respectively. Accuracy can be calculated using the following formula: Accuracy=(TP+TN)/(TP+TN+FP+FN).

Precision can be measured by the percentage of subjects who test positive that are true positives and not false positives. Precision can be calculated using the following formula: precision=TP/(TP+FP).

Classifications can be provided to a subject for example, in the form of recommendations. In one embodiment, the recommendations include a positive recommendation to administer a therapeutic intervention, e.g., a chemotherapy drug.

Individual features may be found to contribute more or less to making an inference. Such significant features can be determined, for example, by leaving them out of a training data set and determining the deterioration in predictive ability of the ultimate models. Also, to the extent statistical analysis generates a plurality of predictive models, comparison of such models can show certain features present in many models.

### A. Companion Diagnostic

Also provided herein are methods for using a companion diagnostic to infer response by a subject (e.g., will or will not respond positively or degree of response) to a therapeutic intervention for oral cancer. A companion diagnostic is an in vitro diagnostic test or device that provides information relevant to the safe and effective use of a corresponding therapeutic intervention, a therapy or adjuvant therapy. Such methods can infer possible adverse reactions to a therapeutic intervention or can infer responsiveness to a therapeutic intervention. Such inferences may include schedule, dose, discontinuation, or combinations of therapeutic agents. In some embodiments, the therapeutic intervention is selected by measuring one or more biomarkers in the subject.

Companion diagnostics can be developed by generating a dataset that includes subjects that are responsive to and nonresponsive to a particular therapeutic intervention. The dataset will further include nucleic acid sequence information derived from a biological sample comprising an oral microbiome of each subject. The dataset can be subject to statistical analysis to identify features, e.g. biomarkers, useful in inferring responsiveness. In some embodiments, the data set is used as a training dataset to train a machine learning algorithm to generate a classification model to classify a subject as responsive or nonresponsive to the particular therapeutic intervention.

The therapeutic intervention can be a primary intervention or an adjuvant therapy for the oral cancer. In adjuvant therapy is an additional therapeutic intervention given after a primary therapeutic intervention to lower the risk that the oral cancer will recur. Adjuvant therapies can include, for example, chemotherapy, radiation therapy, hormone therapy, targeted therapy, or biological therapy.

B. Microbiome Features Associated with Oral Cancer

### 1. Microbiome and KO Features

Table 1 identifies microbial taxa and gene orthologs (e.g., microbial) (identified as KEGG orthologs) associated with oral cancer. The table indicates whether the association is positive (“+”) or negative (“−”). A classification model or rule to infer oral cancer in a subject can a feature set that includes one or more of these markers as features. A variety of combinations of features are possible. These include, without limitation, feature sets including at least, exactly or no more than any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 features selected from the features of Table 1. In another embodiment, all, some or none of the features selected from the features of Table 1 are positively associated with oral cancer. In another embodiment, all, some or none of the features selected from the features of Table 1 are negatively associated with oral cancer. In another embodiment, all, some or none of the features selected from the features of Table 1 are taxonomic features, including features that only positively associated with oral cancer, only negatively associated with oral cancer or a combination of positively and negatively associated features. In another embodiment, all, some or none of the features selected from the features of Table 1 are KEGG ortholog features, including features that only positively associated with oral cancer, only negatively associated with oral cancer or a combination of positively and negatively associated features. In another embodiment, features from Table 1 include both taxonomic features and KEGG ortholog features, including features that are only positively associated with oral cancer, only negatively associated with oral cancer or a combination of positively and negatively associated features. Each feature functions as a biomarker, that is, a measurable biological analyte associated with the condition in question.

TABLE 1

| Feature | Class | Association |
|---|---|---|
| Actinomyces gerencseriae | Taxonomic | Positive |
| | Category | |
| Actinomyces sp. ICM54 | Taxonomic | Positive |
| | Category | |
| Actinomyces sp. oral taxon 170 | Taxonomic | Positive |
| | Category | |
| Actinomyces sp. oral taxon 172 | Taxonomic | Positive |
| | Category | |
| Actinomyces sp. oral taxon 181 | Taxonomic | Positive |
| | Category | |
| Actinomyces sp. oral taxon 849 | Taxonomic | Positive |
| | Category | |
| Actinomyces urogenitalis | Taxonomic | Positive |
| | Category | |
| Alloprevotella rava | Taxonomic | Positive |
| | Category | |
| Alloscardovia omnicolens | Taxonomic | Positive |
| | Category | |
| Arcanobacterium | Taxonomic | Positive |
| urinimassiliense | Category | |
| Bifidobacterium longum | Taxonomic | Positive |
| | Category | |
| Capnocytophaga gingivalis | Taxonomic | Positive |
| | Category | |
| Capnocytophaga sp. oral taxon | Taxonomic | Positive |
| 878 | Category | |
| Corynebacterium | Taxonomic | Positive |
| argentoratense | Category | |
| Eikenella corrodens | Taxonomic | Positive |
| | Category | |
| Haemophilus sp. CCUG 66565 | Taxonomic | Positive |
| | Category | |
| Lactobacillus fermentum | Taxonomic | Positive |
| | Category | |
| Mycoplasma salivarium | Taxonomic | Positive |
| | Category | |
| Parvimonas sp. oral taxon 110 | Taxonomic | Positive |
| | Category | |
| Porphyromonas sp. oral taxon | Taxonomic | Positive |
| 278 | Category | |
| Prevotella buccae | Taxonomic | Positive |
| | Category | |
| Rhodococcus sp. 008 | Taxonomic | Positive |
| | Category | |
| Rothia aeria | Taxonomic | Positive |
| | Category | |
| Rothia sp. HMSC036D11 | Taxonomic | Positive |
| | Category | |
| Rothia sp. HMSC061E04 | Taxonomic | Positive |
| | Category | |
| Rothia sp. HMSC062F03 | Taxonomic | Positive |
| | Category | |
| Rothia sp. HMSC062H08 | Taxonomic | Positive |
| | Category | |
| Rothia sp. HMSC064D08 | Taxonomic | Positive |
| | Category | |
| Rothia sp. HMSC069C01 | Taxonomic | Positive |
| | Category | |
| Selenomonas sp. CM52 | Taxonomic | Positive |
| | Category | |
| Selenomonas sp. oral taxon | Taxonomic | Positive |
| 126 | Category | |
| Selenomonas sp. oral taxon | Taxonomic | Positive |
| 136 | Category | |
| Selenomonas sputigena | Taxonomic | Positive |
| | Category | |
| Staphylococcus pasteuri | Taxonomic | Positive |
| | Category | |
| Streptococcus mitis | Taxonomic | Positive |
| | Category | |
| Streptococcus porcinus | Taxonomic | Positive |
| | Category | |
| Streptococcus sp. 343_SSPC | Taxonomic | Positive |
| | Category | |
| Streptococcus sp. oral taxon | Taxonomic | Positive |
| 056 | Category | |
| Treponema medium | Taxonomic | Positive |
| | Category | |
| Treponema sp. 0MZ 838 | Taxonomic | Positive |
| | Category | |
| Veillonella atypica | Taxonomic | Positive |
| | Category | |
| Xylanimonas cellulosilytica | Taxonomic | Positive |
| | Category | |
| K00163 | KEGG Ortholog | Positive |
| K00313 | KEGG Ortholog | Positive |
| K00692 | KEGG Ortholog | Positive |
| K00929 | KEGG Ortholog | Positive |
| K01251 | KEGG Ortholog | Positive |
| K01253 | KEGG Ortholog | Positive |
| K01576 | KEGG Ortholog | Positive |
| K01697 | KEGG Ortholog | Positive |
| K01804 | KEGG Ortholog | Positive |
| K01903 | KEGG Ortholog | Positive |
| K02023 | KEGG Ortholog | Positive |
| K02445 | KEGG Ortholog | Positive |
| K02552 | KEGG Ortholog | Positive |
| K03019 | KEGG Ortholog | Positive |
| K03154 | KEGG Ortholog | Positive |
| K03338 | KEGG Ortholog | Positive |
| K03492 | KEGG Ortholog | Positive |
| K03573 | KEGG Ortholog | Positive |
| K03579 | KEGG Ortholog | Positive |
| K03609 | KEGG Ortholog | Positive |
| K03610 | KEGG Ortholog | Positive |
| K03781 | KEGG Ortholog | Positive |
| K05692 | KEGG Ortholog | Positive |
| K05799 | KEGG Ortholog | Positive |
| K05825 | KEGG Ortholog | Positive |
| K06076 | KEGG Ortholog | Positive |
| K06200 | KEGG Ortholog | Positive |
| K06603 | KEGG Ortholog | Positive |
| K07289 | KEGG Ortholog | Positive |
| K07343 | KEGG Ortholog | Positive |
| K07678 | KEGG Ortholog | Positive |
| K08982 | KEGG Ortholog | Positive |
| K09766 | KEGG Ortholog | Positive |
| K09788 | KEGG Ortholog | Positive |
| K10546 | KEGG Ortholog | Positive |
| K10547 | KEGG Ortholog | Positive |
| K12452 | KEGG Ortholog | Positive |
| K13276 | KEGG Ortholog | Positive |
| K13276 | KEGG Ortholog | Positive |
| K13497 | KEGG Ortholog | Positive |
| K13922 | KEGG Ortholog | Positive |
| Actinobaculum sp. oral taxon | Taxonomic | Negative |
| 183 | Category | |
| Actinobaculum suis | Taxonomic | Negative |
| | Category | |
| Actinomyces cardiffensis | Taxonomic | Negative |
| | Category | |
| Actinomyces johnsonii | Taxonomic | Negative |
| | Category | |
| Actinomyces massiliensis | Taxonomic | Negative |
| | Category | |
| Actinomyces sp. oral taxon 448 | Taxonomic | Negative |
| | Category | |
| Actinomyces sp. oral taxon 848 | Taxonomic | Negative |
| | Category | |
| Aggregatibacter | Taxonomic | Negative |
| actinomycetecomitans | Category | |
| Aggregatibacter aphrophilus | Taxonomic | Negative |
| | Category | |
| Cardiobacterium hominis | Taxonomic | Negative |
| | Category | |
| Corynebacterium matruchotii | Taxonomic | Negative |
| | Category | |
| Entamoeba nuttalli | Taxonomic | Negative |
| | Category | |
| Kocuria kristinae | Taxonomic | Negative |
| | Category | |
| Leptotrichia buccalis | Taxonomic | Negative |
| | Category | |
| Mogibacterium diversum | Taxonomic | Negative |
| | Category | |
| Neisseria cinerea | Taxonomic | Negative |
| | Category | |
| Neisseria sp. HMSC077D05 | Taxonomic | Negative |
| | Category | |
| Ottowia sp. oral taxon 894 | Taxonomic | Negative |
| | Category | |
| Porphyromonas endodontalis | Taxonomic | Negative |
| | Category | |
| Prevotella loescheii | Taxonomic | Negative |
| | Category | |
| Prevotella sp. oral taxon 473 | Taxonomic | Negative |
| | Category | |
| Propionibacterium australiense | Taxonomic | Negative |
| | Category | |
| Streptococcus cristatus | Taxonomic | Negative |
| | Category | |
| Streptococcus australis | Taxonomic | Negative |
| | Category | |
| Streptococcus lutetiensis | Taxonomic | Negative |
| | Category | |
| Streptococcus mutans | Taxonomic | Negative |
| | Category | |
| Streptococcus phage YMC- | Taxonomic | Negative |
| 2011 | Category | |
| Streptococcus salivarius | Taxonomic | Negative |
| | Category | |
| Streptococcus sobrinus | Taxonomic | Negative |
| | Category | |
| Streptococcus sp. F0442 | Taxonomic | Negative |
| | Category | |
| Streptococcus sp. HPH0090 | Taxonomic | Negative |
| | Category | |
| Streptococcus sp. NPS 308 | Taxonomic | Negative |
| | Category | |
| Streptococcus timonensis | Taxonomic | Negative |
| | Category | |
| Tannerella forsythia | Taxonomic | Negative |
| | Category | |
| K00004 | KEGG Ortholog | Negative |
| K00045 | KEGG Ortholog | Negative |
| K00068 | KEGG Ortholog | Negative |
| K00799 | KEGG Ortholog | Negative |
| K00853 | KEGG Ortholog | Negative |
| K00961 | KEGG Ortholog | Negative |
| K00986 | KEGG Ortholog | Negative |
| K01523 | KEGG Ortholog | Negative |
| K01791 | KEGG Ortholog | Negative |
| K01858 | KEGG Ortholog | Negative |
| K02022 | KEGG Ortholog | Negative |
| K02315 | KEGG Ortholog | Negative |
| K02660 | KEGG Ortholog | Negative |
| K02909 | KEGG Ortholog | Negative |
| K02970 | KEGG Ortholog | Negative |
| K03019 | KEGG Ortholog | Negative |
| K03557 | KEGG Ortholog | Negative |
| K03837 | KEGG Ortholog | Negative |
| K03897 | KEGG Ortholog | Negative |
| K04026 | KEGG Ortholog | Negative |
| K04061 | KEGG Ortholog | Negative |
| K04756 | KEGG Ortholog | Negative |
| K04786 | KEGG Ortholog | Negative |
| K05523 | KEGG Ortholog | Negative |
| K05912 | KEGG Ortholog | Negative |
| K06423 | KEGG Ortholog | Negative |
| K07272 | KEGG Ortholog | Negative |
| K07339 | KEGG Ortholog | Negative |
| K07441 | KEGG Ortholog | Negative |
| K07443 | KEGG Ortholog | Negative |
| K07485 | KEGG Ortholog | Negative |
| K07492 | KEGG Ortholog | Negative |
| K07697 | KEGG Ortholog | Negative |
| K08159 | KEGG Ortholog | Negative |
| K09810 | KEGG Ortholog | Negative |
| K10947 | KEGG Ortholog | Negative |
| K10954 | KEGG Ortholog | Negative |
| K13012 | KEGG Ortholog | Negative |
| K14327 | KEGG Ortholog | Negative |

In certain embodiments, the features used in the model include one or more features selected from Actinobaculum sp. oral taxon 183, Actinomyces massiliensis, Actinomyces sp. oral taxon 448, Alloscardovia omnicolens, Selenomonas sp. CM52, Mycoplasma salivarium, Parvimonas sp. oral taxon 110, Rothia sp. HMSC062H08, K01697, K12452, Actinomyces johnsonii, Prevotella loescheii, Streptococcus cristatus, Streptococcus sobrinus, Streptococcus sp. HPH0090, Tannerella forsythia, and K02909.

### 2. Microbiome, KO and Human Gene Features

Features used by a classification algorithm to infer presence of oral cancer can include a combination of microbial taxa activity scores, microbial KO activity scores, and host gene activity scores. Exemplary features are presented in Tables 2, 3 and 4. In the tables, model coefficient indicates degree of correlation with oral cancer. Greater absolute values indicate higher correlation. Negative and positive scores indicate, respectively, down or up amount of a taxon, or regulation or activity or a KO or gene, compared with control.

Table 2 shows 88 expressed human genes that can be used in a model.

TABLE 2

| Serial | | | |
|---|---|---|---|
| number | Gene ID | Gene name | Model coefficient |
| 1 | ENSG00000114316 | USP4 | −0.11557 |
| 2 | ENSG00000111679 | PTPN6 | −0.10833 |
| 3 | ENSG00000108582 | CPD | −0.10786 |
| 4 | ENSG00000188994 | ZNF292 | −0.10284 |
| 5 | ENSG00000127914 | AKAP9 | −0.0985 |
| 6 | ENSG00000169429 | CXCL8 | 0.09408 |
| 7 | ENSG00000138688 | KIAA1109 | −0.09094 |
| 8 | ENSG00000104093 | DMXL2 | −0.08969 |
| 9 | ENSG00000228253 | MT-ATP8 | −0.08794 |
| 10 | ENSG00000110367 | DDX6 | −0.08734 |
| 11 | ENSG00000095787 | WAC | −0.08594 |
| 12 | ENSG00000101745 | ANKRD12 | −0.08483 |
| 13 | ENSG00000125733 | TRIP10 | −0.08465 |
| 14 | ENSG00000173575 | CHD2 | −0.08183 |
| 15 | ENSG00000145819 | ARHGAP26 | −0.08136 |
| 16 | ENSG00000143631 | FLG | −0.07881 |
| 17 | ENSG00000136694 | IL36A | −0.07627 |
| 18 | ENSG00000133961 | NUMB | −0.07625 |
| 19 | ENSG00000158615 | PPP1R15B | −0.07599 |
| 20 | ENSG00000113648 | MACROH2A1 | −0.07527 |
| 21 | ENSG00000181617 | FDCSP | −0.07455 |
| 22 | ENSG00000134909 | ARHGAP32 | 0.07336 |
| 23 | ENSG00000163659 | TIPARP | −0.07194 |
| 24 | ENSG00000131503 | ANKHD1 | −0.07015 |
| 25 | ENSG00000163216 | SPRR2D | −0.06769 |
| 26 | ENSG00000122862 | SRGN | 0.06769 |
| 27 | ENSG00000172331 | BPGM | −0.06718 |
| 28 | ENSG00000124831 | LRRFIP1 | 0.06706 |
| 29 | ENSG00000166145 | SPINT1 | −0.06542 |
| 30 | ENSG00000008083 | JARID2 | 0.06353 |
| 31 | ENSG00000064932 | SBNO2 | 0.06298 |
| 32 | ENSG00000182795 | C1orf116 | 0.06245 |
| 33 | ENSG00000089159 | PXN | 0.0623 |
| 34 | ENSG00000179218 | CALR | 0.05809 |
| 35 | ENSG00000058272 | PPP1R12A | −0.0574 |
| 36 | ENSG00000066336 | SPI1 | 0.05285 |
| 37 | ENSG00000128016 | ZFP36 | 0.05217 |
| 38 | ENSG00000135052 | GOLM1 | 0.05015 |
| 39 | ENSG00000105374 | NKG7 | 0.04971 |
| 40 | ENSG00000265972 | TXNIP | 0.04583 |
| 41 | ENSG00000197870 | PRB3 | 0.04276 |
| 42 | ENSG00000123689 | G0S2 | 0.04252 |
| 43 | ENSG00000115216 | NRBP1 | 0.04227 |
| 44 | ENSG00000143226 | FCGR2A | 0.04125 |
| 45 | ENSG00000078369 | GNB1 | 0.04062 |
| 46 | ENSG00000087128 | TMPRSS11E | −0.04057 |
| 47 | ENSG00000119922 | IFIT2 | 0.04023 |
| 48 | ENSG00000241794 | SPRR2A | −0.0398 |
| 49 | ENSG00000163739 | CXCL1 | 0.03842 |
| 50 | ENSG00000255398 | HCAR3 | 0.03778 |
| 51 | ENSG00000166317 | SYNPO2L | 0.03654 |
| 52 | ENSG00000164830 | OXR1 | 0.03652 |
| 53 | ENSG00000063177 | RPL18 | 0.03408 |
| 54 | ENSG00000198853 | RUSC2 | 0.03389 |
| 55 | ENSG00000124942 | AHNAK | −0.03344 |
| 56 | ENSG00000216490 | IFI30 | 0.03301 |
| 57 | ENSG00000125503 | PPP1R12C | 0.0318 |
| 58 | ENSG00000160888 | IER2 | 0.03166 |
| 59 | ENSG00000151893 | CACUL1 | 0.03047 |
| 60 | ENSG00000108298 | RPL19 | 0.02844 |
| 61 | ENSG00000173821 | RNF213 | 0.02779 |
| 62 | ENSG00000087086 | FTL | 0.02611 |
| 63 | ENSG00000124102 | PI3 | 0.02425 |
| 64 | ENSG00000043462 | LCP2 | 0.02413 |
| 65 | ENSG00000100292 | HMOX1 | 0.02326 |
| 66 | ENSG00000067225 | PKM | 0.02137 |
| 67 | ENSG00000078618 | NRDC | 0.02073 |
| 68 | ENSG00000092199 | HNRNPC | 0.01947 |
| 69 | ENSG00000148341 | SH3GLB2 | 0.01872 |
| 70 | ENSG00000134531 | EMP1 | −0.01858 |
| 71 | ENSG00000189337 | KAZN | 0.01615 |
| 72 | ENSG00000198830 | HMGN2 | 0.01544 |
| 73 | ENSG00000198771 | RCSD1 | 0.01531 |
| 74 | ENSG00000162191 | UBXN1 | 0.01372 |
| 75 | ENSG00000184922 | FMNL1 | 0.01292 |
| 76 | ENSG00000105388 | CEACAM5 | 0.01131 |
| 77 | ENSG00000186081 | KRT5 | 0.01118 |
| 78 | ENSG00000198858 | R3HDM4 | 0.01066 |
| 79 | ENSG00000170348 | TMED10 | −0.00922 |
| 80 | ENSG00000091317 | CMTM6 | 0.00825 |
| 81 | ENSG00000197006 | METTL9 | 0.00817 |
| 82 | ENSG00000005020 | SKAP2 | 0.00635 |
| 83 | ENSG00000157601 | MX1 | −0.00586 |
| 84 | ENSG00000163346 | PBXIP1 | 0.00544 |
| 85 | ENSG00000118503 | TNFAIP3 | −0.00272 |
| 86 | ENSG00000089820 | ARHGAP4 | 0.00251 |
| 87 | ENSG00000179820 | MYADM | 0.00118 |
| 88 | ENSG00000034510 | TMSB10 | 0.00111 |

Table 3 shows 110 active microbial species that can be used in a model.

TABLE 3
The 110 active species features in the final model

| Serial | | |
|---|---|---|
| number | Species name | Model coefficient |
| 1 | Corynebacterium matruchotii | −0.09455 |
| 2 | Saccharomyces sp. ‘boulardii’ | −0.08952 |
| 3 | Tannerella forsythia | −0.0871 |
| 4 | Actinomyces sp. oral taxon 180 | 0.08283 |
| 5 | Rothia sp. HMSC078H08 | 0.08053 |
| 6 | Streptococcus mutans | −0.07751 |
| 7 | Campylobacter sp. 10_1_50 | −0.07604 |
| 8 | Prevotella sp. oral taxon 472 | −0.0748 |
| 9 | Porphyromonas endodontalis | −0.07454 |
| 10 | Ralstonia sp. MD27 | −0.07117 |
| 11 | Gemella morbillorum | 0.06892 |
| 12 | Ochrobactrum anthropi | 0.06864 |
| 13 | Campylobacter concisus | −0.06862 |
| 14 | Leucobacter chironomi | 0.06695 |
| 15 | Capnocytophaga sp. ChDC OS43 | 0.06538 |
| 16 | Prevotella loescheii | −0.06373 |
| 17 | Rothia sp. HMSC062F03 | 0.05691 |
| 18 | Actinomyces johnsonii | −0.05261 |
| 19 | Actinobaculum sp. oral taxon 183 | −0.05119 |
| 20 | Actinomyces massiliensis | −0.04904 |
| 21 | Prevotella nanceiensis | −0.04837 |
| 22 | Capnocytophaga sp. oral taxon | 0.04717 |
| | 329 | |
| 23 | Neisseria polysaccharea | −0.04502 |
| 24 | Actinomyces sp. oral taxon 170 | −0.04475 |
| 25 | Bifidobacterium reuteri | 0.04413 |
| 26 | Actinomyces viscosus | −0.04364 |
| 27 | Selenomonas sp. CM52 | 0.04296 |
| 28 | Oribacterium parvum | −0.04253 |
| 29 | Leptotrichia hofstadii | −0.04057 |
| 30 | Peptoniphilus sp. oral taxon 836 | 0.03966 |
| 31 | Fusobacterium sp. oral taxon 370 | 0.03855 |
| 32 | Streptococcus vestibularis | −0.03817 |
| 33 | Actinomyces sp. HMSC075C01 | −0.038 |
| 34 | Selenomonas noxia | −0.03714 |
| 35 | Actinomyces sp. oral taxon 849 | −0.03595 |
| 36 | Streptococcus sp. 343_SSPC | −0.03435 |
| 37 | Actinomyces sp. Marseille-P2985 | −0.03204 |
| 38 | Alloscardovia omnicolens | 0.03202 |
| 39 | Prevotella sp. oral taxon 299 | −0.0315 |
| 40 | Streptococcus sp. 1171_SSPC | −0.03104 |
| 41 | Streptococcus sp. 400_SSPC | −0.03008 |
| 42 | Fusobacterium sp. OBRC1 | 0.02958 |
| 43 | Actinomyces sp. oral taxon 877 | −0.02949 |
| 44 | Rothia aeria | −0.02941 |
| 45 | Streptococcus anginosus | 0.02817 |
| 46 | Eikenella corrodens | 0.02815 |
| 47 | Streptococcus milleri | 0.02809 |
| 48 | Bifidobacterium sp. | 0.02809 |
| | 12_1_47BFAA | |
| 49 | Actinomyces sp. oral taxon 448 | −0.02733 |
| 50 | Cardiobacterium hominis | −0.02657 |
| 51 | Haemophilus sp. HMSC61B11 | −0.02591 |
| 52 | Streptococcus sp. HMSC034E12 | 0.02551 |
| 53 | Actinomyces sp. oral taxon 171 | −0.02476 |
| 54 | Actinomyces gerencseriae | −0.02367 |
| 55 | Streptococcus sp. HMSC066F01 | 0.02345 |
| 56 | Haemophilus sp. HMSC71H05 | −0.02255 |
| 57 | Streptococcus viridans | 0.02247 |
| 58 | Mogibacterium diversum | −0.02242 |
| 59 | Streptococcus sanguinis | −0.02089 |
| 60 | Abiotrophia sp. HMSC24B09 | −0.02078 |
| 61 | Fusobacterium sp. HMSC064B11 | 0.01874 |
| 62 | Rothia sp. HMSC036D11 | −0.01852 |
| 63 | Lactobacillus fermentum | 0.01814 |
| 64 | Actinomyces sp. S6-Spd3 | −0.01812 |
| 65 | Streptococcus sp. HMSC072G04 | −0.01781 |
| 66 | Streptococcus sp. HMSC062D07 | −0.01703 |
| 67 | Corynebacterium durum | −0.01692 |
| 68 | Haemophilus sp. HMSC073C03 | −0.01655 |
| 69 | Streptococcus timonensis | −0.01631 |
| 70 | Bifidobacterium longum | 0.0159 |
| 71 | Streptococcus sp. I-G2 | 0.01567 |
| 72 | Leptotrichia wadei | −0.01542 |
| 73 | Bifidobacterium breve | 0.01528 |
| 74 | Streptococcus sp. HMSC065C01 | −0.0151 |
| 75 | Streptococcus sp. I-P16 | −0.01432 |
| 76 | Fusobacterium nucleatum | 0.01382 |
| 77 | Streptococcus sp. HMSC072D03 | −0.01301 |
| 78 | Rothia sp. HMSC064D08 | −0.01277 |
| 79 | Lactobacillus crispatus | 0.01168 |
| 80 | Actinomyces sp. oral taxon 175 | −0.01136 |
| 81 | Haemophilus sp. HMSC061E01 | −0.01085 |
| 82 | Veillonella sp. oral taxon 158 | −0.0107 |
| 83 | Streptococcus constellatus | 0.00982 |
| 84 | Streptococcus sp. AS20 | 0.0096 |
| 85 | Streptococcus sp. F0442 | 0.00942 |
| 86 | Rothia sp. HMSC071F11 | 0.00881 |
| 87 | Streptococcus sp. HMSC10E12 | 0.00833 |
| 88 | Rothia dentocariosa | −0.00829 |
| 89 | Capnocytophaga sputigena | 0.00828 |
| 90 | Oribacterium sinus | 0.00786 |
| 91 | Streptococcus parasanguinis | −0.00761 |
| 92 | Gemella sanguinis | −0.00735 |
| 93 | Streptococcus sp. A12 | −0.00727 |
| 94 | Actinomyces sp. ICM47 | −0.0071 |
| 95 | Streptococcus sp. HMSC072C09 | −0.00686 |
| 96 | Rothia sp. HMSC069C01 | −0.00654 |
| 97 | Streptococcus sp. HMSC068F04 | 0.00609 |
| 98 | Streptococcus sp. SR4 | −0.00464 |
| 99 | Rothia sp. HMSC067H10 | 0.00381 |
| 100 | Prevotella melaninogenica | −0.00331 |
| 101 | Leptotrichia sp. oral taxon 215 | 0.00248 |
| 102 | Actinomyces oris | 0.00213 |
| 103 | Streptococcus salivarius | 0.00179 |
| 104 | Prevotella sp. ICM33 | 0.0016 |
| 105 | Streptococcus sp. 449_SSPC | −0.00132 |
| 106 | Bacteroides zoogleoformans | 0.00103 |
| 107 | Streptococcus sp. HMSC064D12 | 0.00101 |
| 108 | Streptococcus cristatus | 0.0008 |
| 109 | Streptococcus sp. HMSC065E03 | −0.00055 |
| 110 | Rothia mucilaginosa | −8.00E−05 |

Table 4 shows 72 active microbial KO functional features that can be used in a model.

TABLE 4

| Serial | | | Model |
|---|---|---|---|
| number | KO ID | KO name | coefficient |
| 1 | K07012 | cas3 | 0.08723 |
| 2 | K00575 | cheR | −0.07702 |
| 3 | K00350 | nqrE | 0.06995 |
| 4 | KO1460 | gsp | −0.06993 |
| 5 | K12830 | SF3B3, SAP130, RSE1 | 0.06823 |
| 6 | K01222 | E3.2.1.86A, celF | 0.06711 |
| 7 | K11710 | troB, mntB, znuC | 0.06536 |
| 8 | K03154 | this | 0.0638 |
| 9 | K05982 | E3.1.21.7, nfi | −0.06154 |
| 10 | K07673 | narX | −0.05694 |
| 11 | K07104 | catE | 0.05519 |
| 12 | K03332 | fruA | −0.05516 |
| 13 | K00248 | ACADS, bcd | 0.05456 |
| 14 | K03091 | SIG3.4 | 0.05263 |
| 15 | K00459 | ncd2, npd | 0.05168 |
| 16 | K10546 | ABC.GGU.S, chvE | 0.05161 |
| 17 | K00372 | nasA | 0.05121 |
| 18 | K03312 | gltS | 0.05098 |
| 19 | K07402 | xdhC | 0.0501 |
| 20 | K06904 | uncharacterized protein | −0.04933 |
| 21 | K02567 | napA | −0.04693 |
| 22 | K07642 | baeS, smeS | 0.04681 |
| 23 | K02198 | ccmF | 0.04677 |
| 24 | K06894 | yfhM | 0.04676 |
| 25 | K09693 | tagH | 0.04461 |
| 26 | K03760 | eptA, pmrC | 0.04352 |
| 27 | K01802 | E5.2.1.8 | 0.04335 |
| 28 | K01457 | atzF | −0.04331 |
| 29 | K03319 | TC.DASS | 0.04154 |
| 30 | K00809 | DHPS, dys | 0.0412 |
| 31 | K02002 | proX | −0.04116 |
| 32 | K00285 | dadA | 0.04113 |
| 33 | K00765 | hisG | −0.04069 |
| 34 | K01804 | araA | 0.0406 |
| 35 | K06423 | sspF | −0.03798 |
| 36 | K15011 | regB, regS, actS | 0.03772 |
| 37 | K00045 | E1.1.1.67, mtlK | −0.03677 |
| 38 | K04019 | eutA | −0.03657 |
| 39 | K03736 | eutC | −0.03591 |
| 40 | K07751 | pepB | −0.03555 |
| 41 | K03314 | nhaB | −0.03531 |
| 42 | K01442 | E3.5.1.24 | 0.03516 |
| 43 | K01668 | E4.1.99.2 | 0.03449 |
| 44 | K00990 | glnD | −0.03385 |
| 45 | K08963 | mtnA | −0.03352 |
| 46 | K00428 | E1.11.1.5 | 0.03347 |
| 47 | K09158 | uncharacterized protein | −0.03328 |
| 48 | K02006 | cbiO | −0.03291 |
| 49 | K01227 | E3.2.1.96 | 0.03262 |
| 50 | K05825 | LYSN | 0.03128 |
| 51 | K05946 | tagA, tarA | −0.03037 |
| 52 | K02653 | pilC | −0.03 |
| 53 | K01697 | CBS | 0.0298 |
| 54 | K00275 | pdxH, PNPO | 0.02973 |
| 55 | K04772 | degQ, hhoA | −0.02937 |
| 56 | K01581 | E4.1.1.17, ODC1, speC, | 0.02905 |
| | | speF | |
| 57 | K08161 | mdtG | 0.02867 |
| 58 | K05801 | djlA | −0.02676 |
| 59 | K03707 | tenA | 0.0253 |
| 60 | K12940 | abgA | −0.02439 |
| 61 | K01069 | E3.1.2.6, gloB | 0.02311 |
| 62 | K07704 | lytS | −0.02271 |
| 63 | K03777 | dld | 0.02218 |
| 64 | K02009 | cbiN | 0.01981 |
| 65 | K06077 | slyB | −0.0187 |
| 66 | K03610 | minC | 0.01806 |
| 67 | K04026 | eutL | −0.0154 |
| 68 | K10804 | tesA | 0.0124 |
| 69 | K03667 | hslU | 0.01096 |
| 70 | K05803 | nlpl | −0.00963 |
| 71 | K03597 | rseA | −0.00588 |
| 72 | K07136 | uncharacterized protein | 0.00388 |

3. Genesets Associated with Oral Cancer

Referring to Table 5, certain biological mechanisms are associated with oral cancer. Activity of taxa, microbial KOs and host genes that are involved in these mechanisms can be used as features in a classification model to infer oral cancer.

i. Pro-Inflammatory Activities Promoting Carcinogenesis

Among the prominent mechanisms of microbial oral carcinogenesis is the bacterial stimulation of chronic inflammation and production of proinflammatory mediators that facilitates cell proliferation, mutagenesis, oncogene activation, and angiogenesis.

Pathogens/pathobionts and their functions The creation of a sustained dysbiotic proinflammatory environment by periodontal bacteria serves to functionally link periodontal disease and oral cancer. Moreover, traditional periodontal pathogens, such as Porphyromonas gingivalis, Fusobacterium nucleatum, and Treponema denticola, are among the species most frequently identified as being enriched in OSCC, and they possess a number of oncogenic properties. Among the pathogens predictive of OSCC, Porphyromonas, Treponema and Fusobacterium have higher abundances in oral swabs of patients with oral cancer. These organisms share the ability to attack and invade oral epithelial cells, and communicate with the host epithelium, and ultimately acquire phenotypes associated with cancer such as inhibition of apoptosis, increased proliferation, and increased migration of epithelial cells. Additionally, emerging properties of structured bacterial communities may increase oncogenic potential, and consortia of P. gingivalis and F. nucleatum are synergistically pathogenic within in vivo oral cancer models.

Interestingly, some species of oral streptococci can antagonize the phenotypes induced oral pathogens indicating functionally specialized roles for commensals and early colonizers in the oral biofilm. A number of top taxa features that are predictive of controls are components of the Viridans streptococci and commensal flora such as Streptococcus milleri (Gossling, 1988), Actinomyces and Campylobacter concisus. C. concisus was associated with the human oral cavity and has been linked with periodontal lesions, including gingivitis and periodontitis. Clinical studies have linked Streptococcus sp. to both caries progression and early childhood caries. S. anginosus is thought to exist in the mouth as a normal flora and to be located mainly in the gingiva and dental plaque, but one study data strongly indicates the implication of S. anginosus infection in carcinogenesis of head and neck squamous cell carcinoma.

LPS Biosynthesis Bacterial outer membrane lipopolysaccharides are entities that mediate proinflammatory immune response and inflammation host cells. LPS regulates gene expression of pro-inflammatory cytokines through activation of toll-like receptor 4 (TLR4) via NF-kB. The ‘0 antigens’, an extremely polymorphic polysaccharide binds to LipidA to form the LPS outer-membrane of Gram-negative bacteria thereby imparting antigenic specificity to the organism. For instance, LPS from Porphyromonas, a positively associated taxa from the OSCC model, is known to activate macrophages and increase NO production of cancer cell lines.

Biofilm and Virulence The OSCC model predicts a number of functional features associated with bacterial virulence as predictive of oral cancer. CheR are sugar transport and chemotaxis associated KOs respectively present in the oral microbes that are deterministic of virulence and pathogenesis. Cas3, member of CRISPR-associated proteins (CRISPR-Cas) system, is found to be predictive of OSCC from the model, CRISPR-Cas is important in biofilm formation, acquisition of resistance genes, DNA repair, regulation of interspecific competition. Tar gene, TagA is involved in the biosynthesis pathway of poly(ribitol phosphate), with potential involvement in capsular polysaccharide synthesis mediated virulence, autolysin regulator LytS, rscC two-component system which is involved in capsular polysaccharide synthesis mediated virulence, eutL involved in ethanolamine utilization and virulence are all features predictive of oral cancer phenotype from the model.

ii. Hydrogen Sulfide Production in OSCC

Sulfide (H2S) Producers and functional activities in OSCC: Hydrogen sulfide (H2S), a gaseous transmitter, is associated with oral periodontitis and is one of the main causes of halitosis and is generally associated with many oral diseases including oral cancer. Hydrogen sulfide promoted oral cancer cell proliferation through activation of the COX2, AKT and ERK1/2 pathways in a dose-dependent manner. Hydrogen sulfide and the enzymes that synthesize it, cystathionine-b-synthase, cystathionine γ-lyase are increased in different human malignancies. The expression of both enzymes and cellular H2S levels increase tumor survival and promote tumor dedifferentiation. Among the taxa, members of the Streptococcus anginosus group, Fusobacterium and Porphyromonas endodontalis are known producers of oral H2S. The KO CBS (cystathionine beta-synthase) is implicated in the production of oral H2S. The sulfide producing bacteria as well as the functional KOs are all positive predictors of OSCC from the model.

iii. Microbial Contribution to Cancer-Specific Energy Metabolism

Sugar metabolism and alternative energy utilization pathways: Cancer cells strongly upregulate glucose uptake and give rise to increased pyruvate. Unlike in normal cells, the pyruvate is not coupled to the mitochondrial tricarboxylic acid (TCA) cycle, instead is shunted to lactate fermentation and kept away from mitochondrial oxidative metabolism. This shift from oxidative phosphorylation toward aerobic glycolysis, even in the presence of oxygen is known as the “Warburg effect”. In cancer cells, the Pentose Phosphate Pathway (PPP) together with glycolysis, coordinates glucose flux and supports the cellular biogenesis of macromolecules such as lipids, DNA and for energy production. An increased PPP flux in human cancer cells is indicative of its role in meeting the bioenergetic demands of cancer cell proliferation and contribution to the Warburg effect. Enzymes such araA (L-arabinose isomerase) involved in pentose interconversion, as well as 6-phospho-beta-glucosidase involved in sugar metabolism, are positively associated features from the model suggest microbial dysregulation of PPP flux in human cancer cells.

Anti-Inflammatory and Antimicrobial mechanism: The commensal bacteria Streptococcus salivarius establishes in the human oral cavity a few hours after birth and remains there as a predominant commensal and as a primary colonizer of biofilms. Upon strong adhesion mediated by the glycosylated surface-exposed proteins like SrpA, S. salivarius promotes innate immunity by suppressing proinflammatory cascades as well as by producing anti-microbial substances like bacteriocins that antagonizes the virulent streptococci involved in tooth decay or pharyngitis or pathogens involved in periodontitis (Kaci et al 2014). Similarly, Streptococcus gordonii, an early colonial member of oral biofilm produces H2O2 to inhibit the growth of competitors, like the mutans streptococci, as well as strict anaerobic middle and later colonizers of the dental biofilm. Interestingly, Veillonella species, possess a putative catalase gene (catA) that mediates resistance to the S. gordonii thereby enabling direct physical interaction (coaggregate) with S. gordonii as well as Fusobacterium nucleatum that are late colonizers of biofilm. It is interesting to note that Fusobacterium and Veillonella are positive predictors of OSCC.

iv. Protein Fermentation as a Tumorigenic Mechanism

Lysine, Cadaverine metabolism and production pathways: Protein fermentation is a favorable condition in the tumor microenvironment as it results in the accumulation of by-products that are resourceful for the cancer cells. Polyamines such as putrescine and spermidine are products of microbial protein fermentation and are implied in cancer initiation and development. Cancer cells accumulate increased concentrations of polyamines by increased uptake via their PTS (Polyamine Transport System) (Palmer et al 2009). production of amino acids such as Lysine synthesis (LYSN), enhanced putrescine production pathways (ornithine decarboxylase) is observed and predictive of oral cancer phenotype.

Microbial Ammonia production pathways: The cellular protein degradation produces ammonia as a by-product. However, the role of ammonia in cancer cells is still not very clear as ammonia is not merely considered a toxic waste product, but is recycled into central amino acid metabolism to maximize nitrogen utilization. The ammonia accumulated in the tumor microenvironment was used directly to generate amino acids through GDH activity. These data show that ammonia not only is a secreted waste product, but a fundamental nitrogen source that can support tumor biomass. Evidence of increased microbial ammonia production is noted from altered narX, gInD, dadA, tenA, pdxH that are positively predictive of OSCC.

v. Tox Burden

The exposure to synthetic chemicals such as dyes, organopesticides and pharmaceuticals increases the toxicity burden of cells that elevates the cancer causing potential in general. Features involved in benzoate degradation, and atrazine degradation is detected from the predictive model for OSCC. Further, traces of acetaldehyde production (ncd2, npd nitronate monooxygenase) KOs are also observed to be predictive of oral cancer.

vi. Antibiotic Resistance

Antibiotic resistance and drug efflux: Microbes such as streptococcus milleri (Han 2001), Prevotella and Fusobacterium species which are known to show antibiotic resistance are predictive of oral cancer phenotype from the model. Fusobacterium nucleatum via. via the TLR4/NF-κB pathway promoted chemoresistance in CRC. Further, other model predicted features mdtB, multidrug efflux pump, and eptA (via. LPS modification) may also potentially contribute to antibiotic resistance.

TABLE 5
Top mechanistic insights implied by the features predictive of OSCC

| | | Functional Microbial | |
|---|---|---|---|
| | Integrative Themes | Features | References |
| 1 | Pro-inflammatory activities promoting carcinogenesis | | |
| | Pathogens/pathobionts | Porphyromonas, and | Bedran, 2012, Han |
| | and their functions | Fusobacterium, | Y W 2016, Zhang 2008, |
| | | Streptococcus cristatus, | Shiga, 2001 |
| | | Streptococcus milleri, | |
| | | Streptococcus anginosus | |
| | LPS Biosynthesis | Porphyromonas | Bedran, 2012, Parks |
| | | endodontalis, | T et al 2015 |
| | | Streptococcus milleri, | |
| | | Streptococcus cristatus, eptA | |
| | Biofilm and Virulence | CheR, yfhM, TesA, | Doan et al 2008, |
| | | Cas3,EutL, PilC | Huang CB, 2012 |
| 2 | Hydrogen Sulfide production in OSCC | | |
| | Sulfide (H2S) Producers | Fusobacterium and | Zhang et al 2016, |
| | and functional activities | Porphyromonas endodontalis, | Patel et al 2017 |
| | in OSCC | ThiS and CBS | |
| 3 | Microbial contribution to cancer-specific energy metabolism | | |
| | Sugar metabolism and | araA, 6-phospho-beta- | Jianrong 2015 |
| | alternative energy | glucosidase | |
| | utilization pathways | | |
| 4 | Protein fermentation as a tumorigenic mechanism | | |
| | Lysine, Cadaverine | LYSN, ornithine | Palmer et al 2009 |
| | metabolism and | decarboxylase, DHPS | |
| | production pathways | | |
| | Microbial Ammonia | narX, glnD, dadA, tenA, | Salvo, 2003, Read |
| | production pathways | pdxH | 2007 |
| 5 | Tox burden | | |
| | Benzaldehyde, arsenite, | ncd2, npd, arsB | Gadda, 2007 |
| | and other carcinogenic | | |
| | toxins | | |
| 6 | Microbial antibiotic resistance in tumorigenesis | | |
| | Antibiotic resistance and | Streptococcus, | Haque, 2019, Zhang, |
| | drug efflux | Fusobacterium nucleatum | 2019 |
| | | mdtB, eptA, | |

### V. Methods of Screening

Diagnostic methods described herein can be used to screen subjects for further testing or for definitive diagnosis. The current standard of care for OSCC screening and diagnosis relies on a physical exam by a healthcare provider, identification of lesion(s), followed by imaging, invasive biopsy and histopathological evaluation. For oral cancer, the most common type is an incisional biopsy which is regarded as the ‘Gold Standard’ for oral cancer diagnosis. A small piece of tissue is cut from the area that appears to be abnormal. A biopsy can be completed in an outpatient setting or the doctor's office if the location and depths of the abnormal tissue is sufficiently accessible and small. While imaging scans may be completed as part of the diagnosing process, the images are intended to direct the biopsy.

Accordingly, a subject can be screened for oral cancer using the methods described herein. A subject who is inferred to have oral cancer by such methods can then be subject to more definitive diagnosis by other standard methods. So, for example, for such a subject, a provider can perform imaging (e.g., to determine the extent of the lesion), biopsy (e.g., incisional biopsy) and histological preparation (e.g., fixing the tissue, sectioning the tissue, staining the tissue) in the process of making a more definitive diagnosis.

### VI. Methods of Treatment

A subject inferred to have oral cancer by the methods disclosed herein may need a therapeutic intervention. Provided herein are methods of treating a subject determined, by the methods disclosed herein, to have an oral cancer with a therapeutic intervention effective to treat the condition.

As used herein, the terms “therapeutic intervention”, “therapy” and “treatment” refer to an intervention that produces a therapeutic effect (e.g., treats) a pathological condition. A therapeutic effect is one that ameliorates, prevents, slows the progression of, delays the onset of symptoms of, improves the condition of (e.g., causes remission of), improves symptoms of, or cures a pathological condition, such as oral cancer.

As used herein, the term “effective” as modifying a therapeutic intervention or treatment (e.g., “therapeutic intervention effective to treat” or “an effective therapeutic intervention” or to amount of a pharmaceutical drug, supplement or food (e.g., “amount effective to treat” or “an effective amount”), refers to a therapeutic intervention or amount of such to produce a therapeutic effect. For example, for the given parameter, a therapeutic intervention effective to treat a condition will show an increase or decrease in the parameter of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

A therapeutic intervention can include, for example surgical removal of cancerous tissue; administration of a chemotherapeutic agent; and administration of a dietary supplement, a food ingredient, or a food that diminishes a dysbiosis in the oral microbiome of the subject associated with the cancer, any of which can alleviate the cancer or its symptoms.

A therapeutic intervention can include, for example, administration of a treatment, administration of a pharmaceutical, or a biologic or nutraceutical substance with therapeutic intent. The response to a therapeutic intervention can be complete or partial. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

Treatments can include administration of therapeutic interventions to re-balance the microbiome toward a taxonomic and/or functional biomarker profile associated with absence of cancer (e.g., associated with health). Such interventions can include administration of therapeutic compositions that reduce the taxa or proteins over-represented in oral cancer and/or encourage the growth of taxa or expression of proteins under-represented in oral cancer. For example, to the extent inflammation is associated with cancer, taxa and gene functions that promote inflammation may be re-balanced toward normal. For example, certain Gram-negative bacteria or production of lipopolysaccharide have been recognized as pro-inflammatory, while certain Clostridia or butyrate producing proteins have been recognized as anti-inflammatory.

One method involves increasing the abundance of an under-represented taxon. This can be achieved by directly providing taxon-specific nutrients to enhance its growth, providing substrates to other taxa that cross-feed the taxon of interest, reducing competing taxa that may inhibit the growth or sequester the nutrients from the taxon of interest, or providing the taxon of interest in the form of a probiotic.

Another method involves reducing the abundance of an over-represented taxon. This can be achieved by depriving the taxon of nutrients, targeting it with bacteriophages, targeting it with the immune system (for example with IgA or IgG antibodies), targeting it with small molecules, increasing the abundance of competing taxa, or reducing the abundance of cross-feeding taxa.

Another method involves reducing the abundance of a microbial function, that is, activity of a KO or a pathway (e.g., a function of Table 5). This can be achieved by reducing the taxon that is expressing the function, reducing the gene expression of the protein(s) involved in the function (by regulatory mechanisms or removal of the substrate), inhibition of the function, or stimulation of the redundant pathways (in the same taxon or another).

Another method involves increasing the abundance of a microbial function, that is, activity of a KO or a pathway (e.g., a function of Table 5). This can be achieved by increasing the taxon that is expressing the function, increasing the gene expression of the protein(s) involved in the function (by regulatory mechanisms or provision of the substrate), stimulation of the function (allosteric effects, post-transcriptional modification), or inhibition of the redundant pathways (in the same taxon or another).

Another method involves preventing the interactions between microorganisms or their molecules (metabolites, nucleic acids, proteins) and human tissue that may support cancer onset or progression. This can be achieved by maintaining a healthy mucosal barrier, reducing inflammation, avoiding detergents in food, avoiding alcohol, avoiding mouthwash, reducing taxa that consume the mucus, increasing the abundance of the taxa that stimulate mucus production, inhibiting human molecules that respond to microbial stimuli.

Another method involves enhancing the interactions between microorganisms or their molecules (metabolites, nucleic acids, proteins) and human tissue that may inhibit cancer onset or progression. Increasing the expression of the human genes that respond to microbial stimuli, increasing microbial taxa or functions, increasing mucus-consuming taxa, increasing the permeability of mucus.

In certain embodiments, after inferring presence of oral cancer in a subject and, optionally, a stage of cancer, the subject is provided with a therapeutic intervention to treat the cancer. Therapeutic interventions for oral cancer include, for example, surgery to remove the cancerous tissue, radiation therapy, chemotherapy, dietary changes, nutritional supplements and combinations of these. Examples include prebiotics (fibers, other molecules), probiotics, bacteriophages, and natural and synthetic small molecules. Providing a therapeutic intervention can include delivering to the subject a package containing a therapeutic composition, e.g., a drug, a food or a dietary supplement. Delivery can be, for example, by common carrier, such as a national postal system, or a private courier service, such as FedEx, UPS, or DHL.

The therapeutic intervention can include administration to a subject a probiotic in an amount to balance a dysbiosis in the subject. For example, described herein are microbial taxa that are over-represented or under-represented compared to normal in oral cancer. The therapeutic intervention can include administering to the subject the microbes that are under-represented, or one or more microbes other than those over-represented in order to re-balance the microbiome toward a healthy profile.

### VII. Computer Systems

Models provided herein can be executed by programmable digital computer.

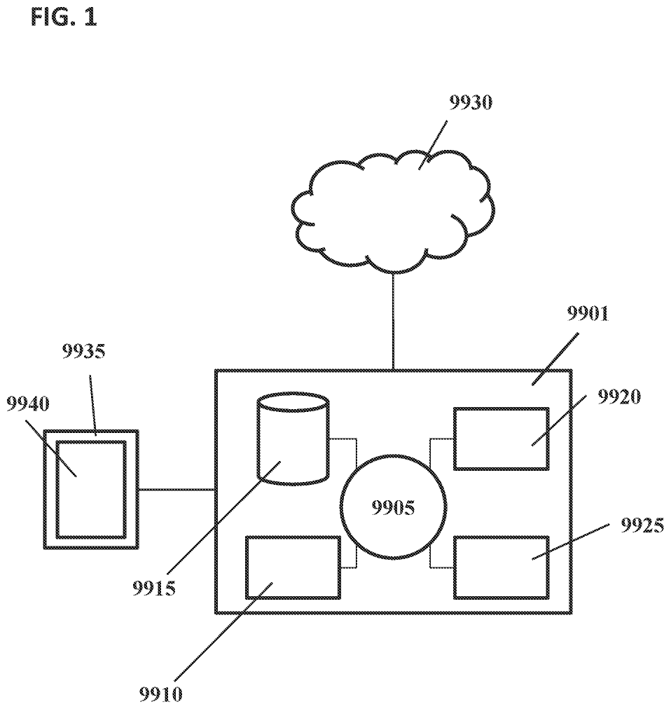

FIG. 1 shows an exemplary computer system. The computer system 9901 includes a central processing unit (CPU, also “processor” and “computer processor” herein) 9905, which can be a single core or multi core processor, or a plurality of processors for parallel processing. The computer system 9901 also includes memory or memory location 9910 (e.g., random-access memory, read-only memory, flash memory), electronic storage unit 9915 (e.g., hard disk), communication interface 9920 (e.g., network adapter) for communicating with one or more other systems, and peripheral devices 9925, such as cache, other memory, data storage and/or electronic display adapters. The computer readable memory 9910, storage unit 9915, interface 9920 and peripheral devices 9925 are in communication with the CPU 9905 through a communication bus (solid lines), such as a motherboard. The storage unit 9915 can be a data storage unit (or data repository) for storing data. The computer system 9901 can be operatively coupled to a computer network (“network”) 9930 with the aid of the communication interface 9920. The network 9930 can be the Internet, an internet and/or extranet, or an intranet and/or extranet that is in communication with the Internet. The network 9930 in some cases is a telecommunication and/or data network. The network 9930 can include one or more computer servers, which can enable distributed computing, such as cloud computing.

The CPU 9905 can execute a sequence of machine-readable instructions, which can be embodied in a program or software. The instructions may be stored in a memory location, such as the computer readable memory 9910. The instructions can be directed to the CPU 9905, which can subsequently program or otherwise configure the CPU 9905 to implement methods of the present disclosure.

The storage unit 9915 can store files, such as drivers, libraries and saved programs. The storage unit 9915 can store user data, e.g., user preferences and user programs. The computer system 9901 in some cases can include one or more additional data storage units that are external to the computer system 9901, such as located on a remote server that is in communication with the computer system 9901 through an intranet or the Internet.

The computer system 9901 can communicate with one or more remote computer systems through the network 9930.

Methods as described herein can be implemented by way of machine (e.g., computer processor) executable code stored on an electronic storage location of the computer system 9901, such as, for example, on the computer readable memory 9910 or electronic storage unit 9915. The machine executable or machine-readable code can be provided in the form of software. During use, the code can be executed by the processor 9905. In some cases, the code can be retrieved from the storage unit 9915 and stored on the memory 9910 for ready access by the processor 9905. In some situations, the electronic storage unit 9915 can be precluded, and machine-executable instructions are stored on memory 9910.

Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks.

The computer system 9901 can include or be in communication with an electronic display 9935 that comprises a user interface (UI) 9940 for providing, for example, input parameters for methods described herein. Examples of UIs include, without limitation, a graphical user interface (GUI) and web-based user interface.

Processes described here can be performed using one or more computer systems that can be networked together. Calculations can be performed in a cloud computing system in which data on the host computer is communicated through the communications network to a cloud computer that performs computations and that communicates, or outputs results to a user through a communications network. For example, nucleic acid sequencing can be performed on sequencing machines located at a user site. The resulting sequence data files can be transmitted to a cloud computing system where the sequence classification algorithm performs one or more operations of the methods described herein. At any step cloud computing system can transmit results of calculations back to the computer operated by the user.

Data can be transmitted electronically, e.g., over the Internet. Electronic communication can be, for example, over any communications network include, for example, a high-speed transmission network including, without limitation, Digital Subscriber Line (DSL), Cable Modem, Fiber, Wireless, Satellite and, Broadband over Powerlines (BPL). Information can be transmitted to a modem for transmission, e.g., wireless or wired transmission, to a computer such as a desktop computer. Alternatively, reports can be transmitted to a mobile device. Reports may be accessible through a subscription program in which a user accesses a website which displays the report. Reports can be transmitted to a user interface device accessible by the user. The user interface device could be, for example, a personal computer, a laptop, a smart phone or a wearable device, e.g., a watch, for example worn on the wrist.

### VIII. Communicating Results in Implementing Wellness/Therapeutic Interventions

Inference models as described herein can be executed on subject data to produce predicted oral cancer and/or recommendations for therapeutic intervention. In one embodiment, after making an inference about a state of oral cancer, the method can comprise developing a model for therapeutic intervention in the subject. The model can comprise, for example, pharmaceutical compositions to administer to the subject to treat the condition. Such a model and be communicated to the subject, for example, transmitting the model and, optionally, the diagnosis, to a user interface of a personal computing device of the subject.

Inferences on a subject's cancer state and/or recommendations for therapeutic intervention can be provided to subjects through an Internet website. A website can be provided which can be accessed by a subject, e.g. a customer, through a password-protected portal. The website can include a clickable icon. Upon clicking the icon, the subject can receive personalized food recommendations. Such inferences and/or recommendations can be displayed on a webpage connected to the clickable icon. Subject can receive at an Internet connected server notification that inferences and/or recommendations for the subject are available.

After wellness/therapeutic interventions are implemented, the effect of these interventions on the subject's condition can be remeasured. Such remeasurements can be used to generate updated inferences and/or recommendations as described herein.

### Examples

A subject's saliva sample is collected in a sample collection and transport kit. The kit includes a saliva collection device that consists of three injection-molded polypropylene components:
The container, where saliva is collected and later shipped;
The funnel/insert which is a single piece that has a dual purpose. It enables a patient to direct the saliva into the tube neatly. The attached cylindrical insert contains the sample preservative that stabilizes RNA.
The cap, which seals the saliva sample inside the container for secure shipping.

Prior to sample collection, the saliva sample collection and transport device has an ambient temperature stability of 12 months. Saliva is deposited into the funnel at the top of the tube. The tube contains a 1.2 mL graduation on the outside wall to ensure an appropriate amount of saliva is collected. Patients are instructed to deposit at least to the 1.2 mL mark (saliva+preservative). The lab process requires a minimum of 175 uL (saliva+preservative). Once sufficient saliva is collected, the funnel is turned counterclockwise, which removes the stem and releases the RNA stabilizer into the tube.

Patients are instructed to cap the tube and shake thoroughly to mix the RNA stabilizer, which preserves RNA in the sample at room temperature for at least 28 days. The secondary container is then placed in a return mailer that further protects the sample.

The RNA stabilizer (1.2 mL per tube) is a commercial product called DNA/RNA Shield from Zymo Research. Note: this same stabilizer is used in Zymo Research's 510(k)-cleared collection device (K202641). This solution both inactivates pathogens and preserves RNA at ambient temperature for prolonged periods without cold-chain. The manufacturer states that “DNA/RNA Shield” viral transport solution has been demonstrated to inactivate Ebola, Influenza, and Herpes Simplex viruses while preserving the integrity of the RNA and DNA for subsequent molecular detection.

Saliva Sample Processing

Once the sample arrives at the laboratory, the lab will visually inspect the tube integrity and approximate volume of the specimen to ensure it is adequate for processing. Each specimen is logged into a LIMS system and if there is more than 1 mL available, it is split into aliquots with any extra aliquots (beyond the 1 for testing) being stored at −80° C. in case repeat testing is necessary (e.g., in the case of an invalid result). The specimen (either fresh or after thawing from −80° C.) are then lysed to release contents using bead beating in a chemical denaturant. This step is performed using the MPBio FastPrep 24 instrument. The lysed specimen is centrifuged to clarify the lysate at 12,000 rpm for 3 minutes. Clarified lysate is transferred to a plate format and diluted with water (1:1).

Total RNA is extracted from clarified lysate using a modified mirVana protocol, which includes on-bead DNA removal by DNase. Total RNA is quantified using the RiboGreen kit, and up to 250 ng of total RNA is transferred to a new plate. Bacterial and human rRNAs are physically removed from the specimen using a subtractive hybridization method. Biotinylated DNA probes complementary to rRNAs are hybridized to the total RNA in a proprietary hybridization buffer. The probe-rRNA complexes are bound to streptavidin magnetic beads. The beads are removed from the solution with a magnet. The remaining RNAs, found in the supernatant, are aspirated and used downstream. Finally, the remaining RNAs are converted into Illumina sequencing libraries using template-switching mechanism with random hexamers for the reverse transcription step.

The patient samples are run using a 96 well tray. To prepare the RNA samples for this high-throughput analysis, each specimen is barcoded with 11 bp dual unique molecular barcodes. During barcoding, PCR is performed with a limited number of cycles and limited primer amounts, leading to an equimolar concentration of each sample library at the end of PCR (due to exhaustion of the primers). Sample libraries are pooled by mixing equal volumes. Sample library pools are purified using AMPure XP beads, which remove buffer components and unincorporated nucleotides. Concentration of each sample library pool is determined using the Qubit 2.0 method with high sensitivity DNA kits.

Sample library pools are sequenced on Illumina NovaSeq 6000 to produce sequencing data.

The raw sequencing data from each flowcell is demultiplexed into FASTQ files corresponding to individual samples and each sample's sequencing reads are then subjected to quality control steps. The quality control passing criteria included a minimum of 1 million reads and 50 strain-level taxa per sample. The remaining high quality paired-end reads are used for detection and quantification of human genes, microbial taxonomies and microbial functions.

For human gene (HG) detection, paired-end reads were mapped to the human genome. Gene expression levels were computed by aggregating transcripts per million estimates per gene using an approach based on Salmon version 1.1.0 (Patro et al., 2017). For taxonomic classification, reads are mapped to a custom catalog derived from genomic sequences from all domains of the phylogenetic tree, namely, bacteria, archaea, eukaryota, and viruses. Taxonomies are identified and their relative activities are calculated at three different taxonomic ranks (genus, species, and strain). To identify and quantify transcriptionally active genes in the microbial community, functional assignments (KOs) are obtained through alignment of the sequencing reads to another custom catalog of Genes (derived from Integrated non-redundant Gene Catalog of the human gut microbiome (IGC) among others) and the Kyoto Encyclopedia of Genes and Genomes (KEGG) databases.

The identified and quantified HGs, species and KOs for a given sample are then provided to the OSCC classifier, which classifies the sample as belonging to the “OSCC class” or the “Not OSCC class” within pre-specified performance criteria.

The final model produced from our V128 BDR model development protocol, which was validated on an independent sample set, encapsulates the following features:

Total number of features: 270
Number of Human Gene features: 88
Number of Species features: 110
Number of KO features: 72

The particular features are provided in Tables 2, 3 and 4.

Bioinformatics

Sequenced data is processed through a cloud-based bioinformatics pipeline and an OSCC classifier.

For developing a model for OSCC classification, the following steps were performed:

1. Following sample processing, perform data quality check for effective sequencing depth, and preprocess the sample data for normalization, computing relative abundance, and removing low prevalence genes;
2. Set up the algorithmic experiments with various combinations of feature sets and hyperparameters;
3. Perform a grid search algorithm by fitting logistic regression models for each feature set and hyperparameter set, cross-validating on the hyperparameter space, and selecting hyperparameter sets that meet the minimum performance criteria;
4. Select the final hyperparameter set based on all relevant performance criteria, and re-train a final model with all available samples.

The classification algorithm was developed and trained on saliva specimens from 945 patients (80 OSCC Positive, 48 OPMD Positive, 12 OPC Positive, and 805 OSCC negative). The OSCC Positive cases were collected from a secondary care center (University Hospital). The patient data also included histopathology reports from Pathologists and Oncologists, spanning early and late stage OSCC. The 805 OSCC negative samples were obtained from a combination primary care centers (which use the previously described standard of care techniques) and individuals self-reporting their cancer status based on their primary care provider's assessment.

In development, numerous different combinations of features (e.g., human genes, microbes) were interrogated to determine which had the best performance. The trained algorithm (or model) was considered to have passed the testing phase if it is able to classify the testing dataset correctly for at least 90% (sensitivity) of the test samples. The performance characteristics of the model (accuracy, specificity, sensitivity, etc.) were then computed using the results from the known test dataset.

Out of the 93 hyperparameter sets (models) that meet the performance constraints, the cross-validation performance were inspected, including ROC-AUC, sensitivity, specificity and the variance of the performance metrics. Viome selected the model that had the highest performance score, defined as the sum of average CV sensitivity and average CV specificity, among the models trained on a feature set containing human genes. The locked-down model, for the independent validation contains a total of 270 features which are used by the classifier for determining the preliminary OSCC status.

Once the model passed the testing phase, the trained classification model was able to take as input the data from an unknown sample and classify it as belonging to the “Oral Cancer class” or the “Not Oral Cancer class” within the desired performance characteristics. At that point, the machine-learnt model is considered to have learned the key properties (or “patterns”) corresponding to Oral Cancer within the training dataset.

The model was validated using saliva samples from 157 subjects (20 OSCC Positive and 137 OSCC Negative).

OSCC Classifier—Molecular Signature

The OSCC Classifier is a model derived from 270 features that included 88 human gene features and 182 microbial features (110 species and 72 KO). The specific features are listed in Tables 2, 3 and 4. This set of 270 features is collectively called the “molecular signature” of patients likely to have OSCC. The features in this molecular signature are associated with molecular processes associated with the biology of cancer.

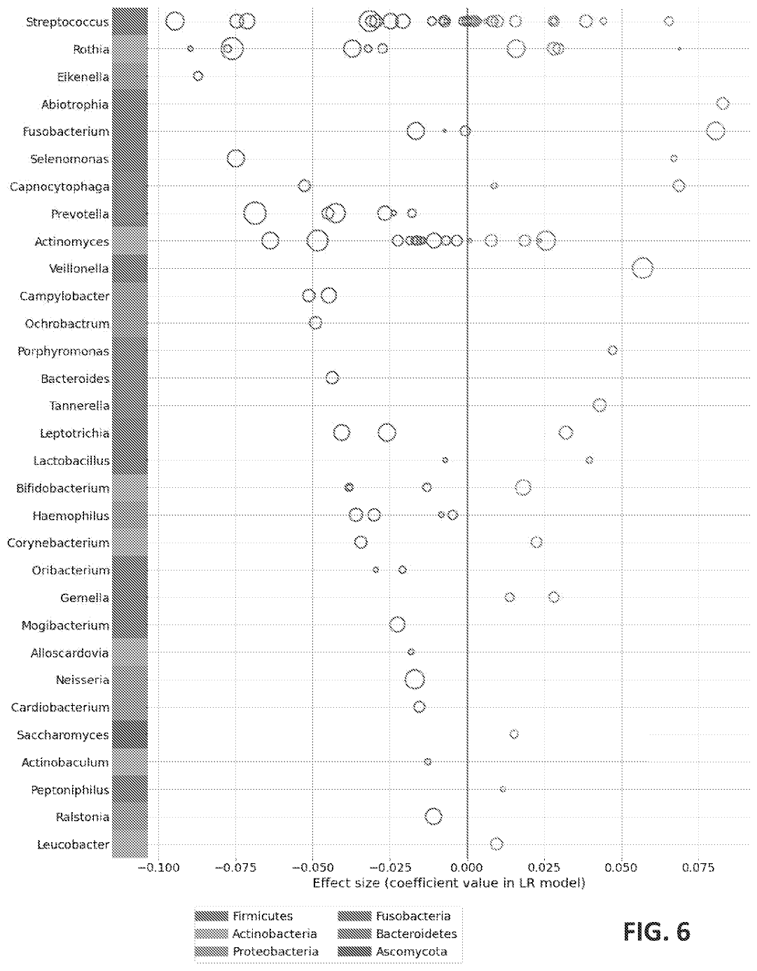

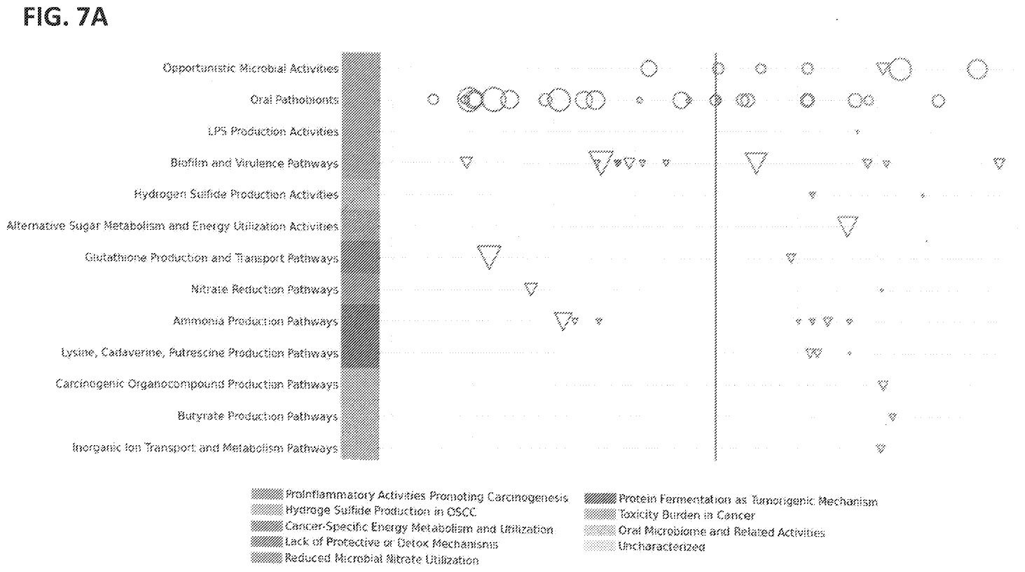

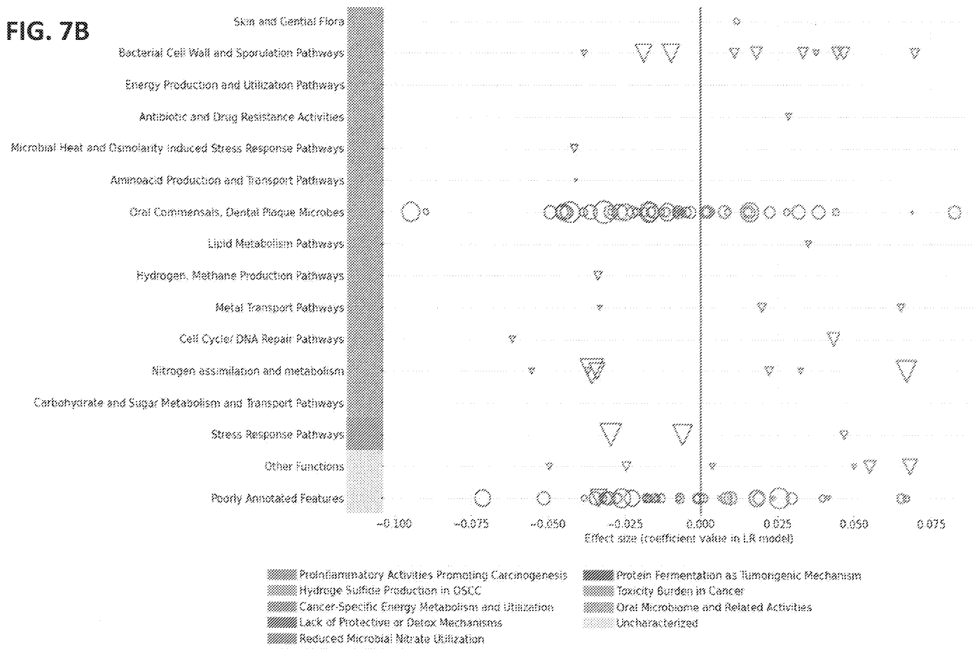

The 88 human genes have a statistically significant overlap with several cancer hallmark genesets such as interferon Gamma, interferon Alpha, KRAS signaling and p53 pathways, with an analysis done via a Gene Set Enrichment Analysis (GSEA) tool. GSEA analysis relies on the enrichment score as the maximum deviation from zero encountered in the random walk; it corresponds to a weighted Kolmogorov-Smirnov-like statistic to compute the overlaps of a curated set from a Molecular Signatures Database (MSigDB) to a new set of genes originating from a new study. MSigDB is a collection of annotated gene sets divided into major collections, representing a universe of biological processes and pathways which are meaningful for insightful interpretation, each based on published experimental findings. This analysis, detailed in Table 5 and FIGS. 6 and 7, shows that the 88 human gene features in our model represent known associations with the biology of cancer.

The 182 microbial features (110 species and 72 KOs listed in Tables 3 and 4) are also collectively consistent with the evidence from a modified polymicrobial synergy and dysbiosis model for bacterial involvement in OSCC. Table 5 and FIGS. 6 and 7 describe the features that are predictive of OSCC and sheds light on some of the mechanisms in oral dysbiosis and periodontal conditions that mediate oral carcinogenesis. The top mechanistic insights implied by these microbial features include pro-inflammatory activities promoting carcinogenesis, hydrogen Sulfide production in OSCC, microbial contribution to cancer-specific energy metabolism, protein fermentation as a tumorigenic mechanism, toxicity burden, and microbial antibiotic resistance in tumorigenesis.

Gene set enrichment analysis was performed to compute the overlap between the gene set found in our model consisting of 88 genes and the MSigDB which is a curated collection of over 30,000 gene sets.

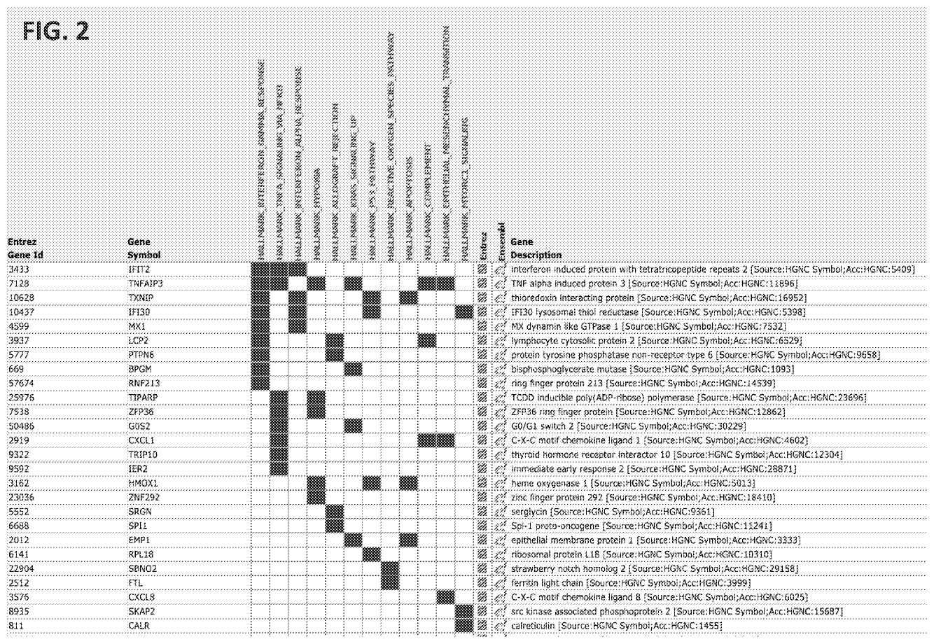

FIG. 2 shows the genesets with highest statistically significant overlap (FDR q-value <=0.05) in the 50 Hallmark genesets. Hallmark agenda sets include: interferon gamma response, TNF alpha signaling via NFKB, interferon alpha response, hypoxia, allograft rejection, KRAS signaling up, p53 pathway, reactive oxygen species pathway, apoptosis, complement, epithelial mesenchymal transition, and MTORC1 signaling. Both interferon Gamma and interferon Alpha genesets show significant overlap, as well as KRAS signaling and p53 pathway.

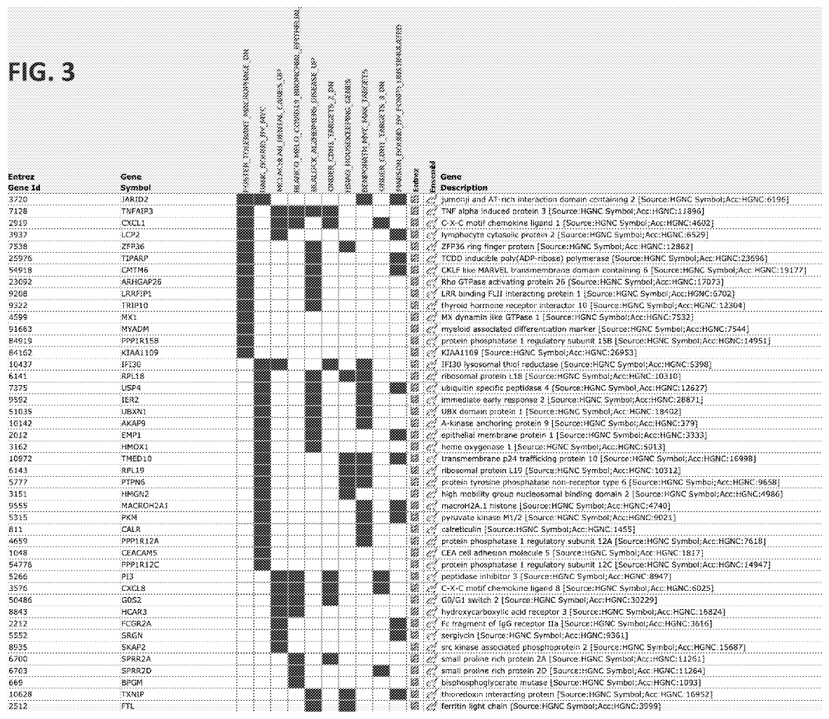

FIG. 3 shows the statistically significant overlap with genesets in the Catalog of Chemical and Genetic perturbations (out of 3358 genesets). Genesets include: Foster Tolerant Macrophage DN, DANG bound by MYC, Mclachlan Dental Caries up, Blanco Melo COVID 19 bronchial epithelial, Blalock Alzheimer's Disease up, under CDH one targets to DNA, HS IAO housekeeping genes, been poor at NYC MA X targets, Onder CDH1 targets 2 DN, and Marson bound by FOXP3 unstimulated. Notably, genes whose promoters are bound by the MYC oncogene are very relevant, and showed up in two overlapping genesets. We also note involvement of the inflammatory processes which is present in genesets such as the Foster-macrophage-related response to lipopolysaccharides (involving TLR genes which broadly inhibit inflammatory response), Blanco-Melo geneset which are upregulated upon epithelial infection with SARS-COV2 as well as genes upregulated in pulpal tissue of dental caries. Two separate signature sets are picked up related to downregulation of genes upon downregulation of E-cadherin (CDH1) tumor suppressor, whose loss is associated with progression in cancer by increasing proliferation, invasion, and/or metastasis.

FIG. 3 shows genesets with statistically significant overlap with Canonical pathways which include 2868 genesets from KEGG, BioCarta and Reactome. Genesets include: reactome formation of the cornified envelope, WP VEGFAVEGFR2 Signaling Pathway, reactome Keratinization, reactome innate immune system.

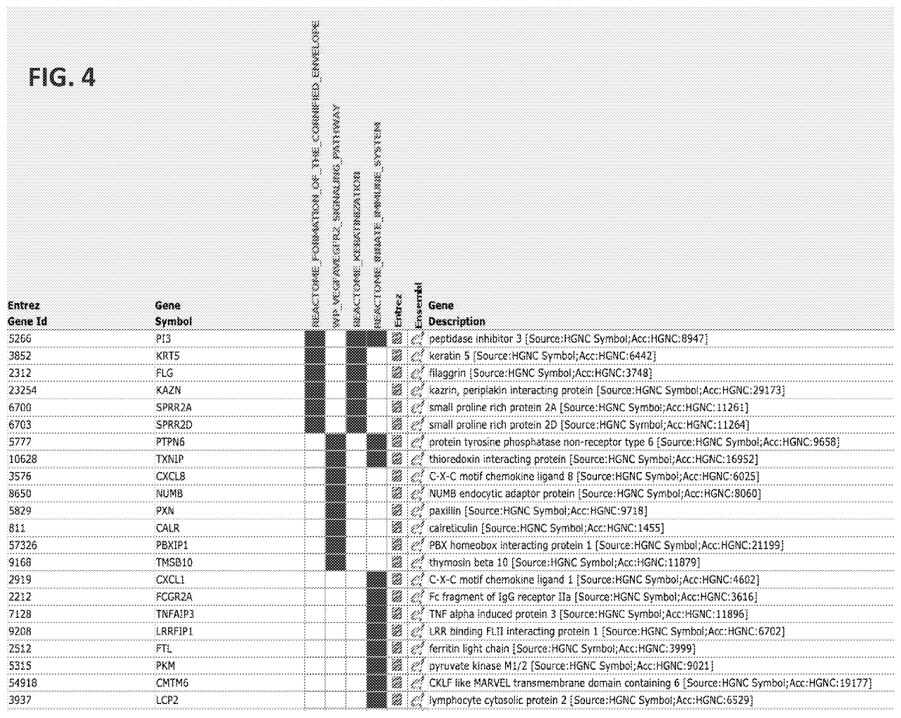

FIG. 4 shows the overlap with oncogenic signature sets. Genesets include: STK33 Nomo up, RPS14 DNLV1 up, p53 DNLV2 up, STK33 up, KRAS lung breast up.V1 up, KRAS.600 up.V1 up, KRAS 600.lung.breast up.V1 up, LEF1 up.V1 up, MEK up.V1 up. Most notably, genesets upregulated upon downregulation of STK33 [Scholl 2009] as well as KRAS, the most commonly mutated oncogene, are prominent.

The Molecular Signatures Database (MSigDB) is a collection of annotated gene sets for use with gene set enrichment (GSEA) software (worldwideweb site: https://gsea-msigdb.org/gsea/msigdb/index.jsp). This method and the accompanying software focuses on groups of genes (genesets) that share a common biological function, location or regulation aspects. GSEA analysis relies on the enrichment score as the maximum deviation from zero encountered in the random walk; it corresponds to a weighted Kolmogorov—Smirnov-like statistic to compute the overlaps of a curated set from MSigDB to a new set of genes originating from a new study. In this manner, we are able to compare a list of genes in our oral cancer study with 31117 gene sets (divided into 9 major collections) in the MSigDB [Liberzon, 2011]. MSigDB represents a universe of biological processes and pathways which are meaningful for insightful interpretation, each based on published experimental findings.

### Exemplary Embodiments

1. A method comprising:

a) providing a biological sample from a subject comprising mouth-sourced cells;
b) sequencing nucleic acids from the sample to produce sequence information;
c) determining, from the sequence information, (1) measures of activity of one or more microbial taxa, (2) measures of activity of one or more microbial gene orthologs, and/or (3) measures of activity of one or more somatic cell genes of the subject, wherein the one or more measures are included in a feature set;
d) executing by computer a classification model that infers, from one or more features in the feature set, a state of oral cancer in the subject.

2. The method of embodiment 1, wherein the biological sample comprises saliva.

3. The method of embodiment 1, wherein the biological sample comprises microbial cells and host cells.

4. The method of embodiment 1, wherein the subject is a human.

5. The method of embodiment 1, wherein the subject is over 50 years of age or has a history of tobacco use.

6. The method of embodiment 1, wherein the mouth-sourced cells comprise an oral microbio and, optionally, somatic cells from the subject.

7. The method of embodiment 6, wherein the somatic cells from the subject comprise cells selected from cheek cells, gum cells and tongue cells.

8. The method of embodiment 1, wherein the nucleic acids sequenced comprise mRNA and the sequence information comprises metatranscriptomic information.

9. The method of embodiment 1, wherein the feature set used by the classification algorithm includes at least: (1) measures of activity of one or more microbial taxa.

10. The method of embodiment 9, wherein the feature set used by the classification algorithm further includes: (2) measures of activity of one or more microbial gene orthologs.

11. The method of embodiment 10, wherein the feature set used by the classification algorithm further includes: (3) measures of activity of one or more host somatic cell genes.

12. The method of embodiment 1, wherein the feature set used by the classification algorithm includes at least two of: (1) measures of activity of one or more microbial taxa, (2) measures of activity of one or more microbial gene orthologs, or (3) measures of activity of one or more somatic cell genes of the subject.

13. The method of embodiment 1, wherein the classification model uses one or more features selected from the features of Table 1.

14. The method of embodiment 1, wherein the classification model uses at least, exactly or no more than any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, or 157 of the features selected from the features of Table 1.

15. The method of embodiment 1, wherein the classification model uses at least, exactly or no more than any of 1, 2, 3, 4, 5, 6, 7, 8, 9, 10, 11, 12, 13, 14, 15, 16, or 17 of the features selected from: Actinobaculum sp. oral taxon 183, Actinomyces massiliensis, Actinomyces sp. oral taxon 448, Alloscardovia omnicolens, Selenomonas sp. CM52, Mycoplasma salivarium, Parvimonas sp. oral taxon 110, Rothia sp. HMSC062H08, K01697, K12452, Actinomyces johnsonii, Prevotella loescheii, Streptococcus cristatus, Streptococcus sobrinus, Streptococcus sp. HPH0090, Tannerella forsythia, and K02909.

16. The method of embodiment 15, wherein the features of Table 1 include one or more microbial taxa features and/or one or more gene ortholog features.

17. The method of embodiment 15, wherein the features of Table 1 include one or more positively associated features and/or one or more negatively associated features.

18. The method of embodiment 1, wherein the classification model uses only features selected from the features of Table 1.

19. The method of embodiment 1, wherein the feature set used by the classification algorithm includes at least 30, at least 50, at least 100, at least 200 or all of the features selected from Tables 2, 3 or 4.

20. The method of embodiment 19, wherein the feature set used by the classification algorithm includes at least 10 microbial taxa features, at least 10 microbial gene ortholog features and at least 10 host cell gene features.

21. The method of embodiment 19, wherein the feature set used by the classification algorithm further includes: mechanism feature, a toxic burden feature (3) measures of activity of one or more host somatic cell genes.

22. The method of embodiment 19, wherein the features of Table 1 include one or more microbial taxa features and/or one or more gene ortholog features.

23. The method of embodiment 19, wherein the features of Table 1 include one or more positively associated features and/or one or more negatively associated features.

24. The method of embodiment 1, wherein the classification model uses only features selected from the features of Tables 2, 3 and 4.

25. The method of embodiment 1, wherein the classification model uses at least, exactly or no more than any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, or 270 of the features selected from the features of Tables 2, 3 or 4.

26. The method of embodiment 1, wherein the feature set used by the classification algorithm includes one or more features selected from a pro-inflammatory activity feature, a hydrogen sulfide production activity feature, a microbial contribution to cancer-specific energy metabolism feature, a protein fermentation as a tumor genic mechanism feature, tox burden feature, and microbial antibiotic resistance in tumorigenesis feature.

27. The method of embodiment 26, wherein the selected features are from Table 5.

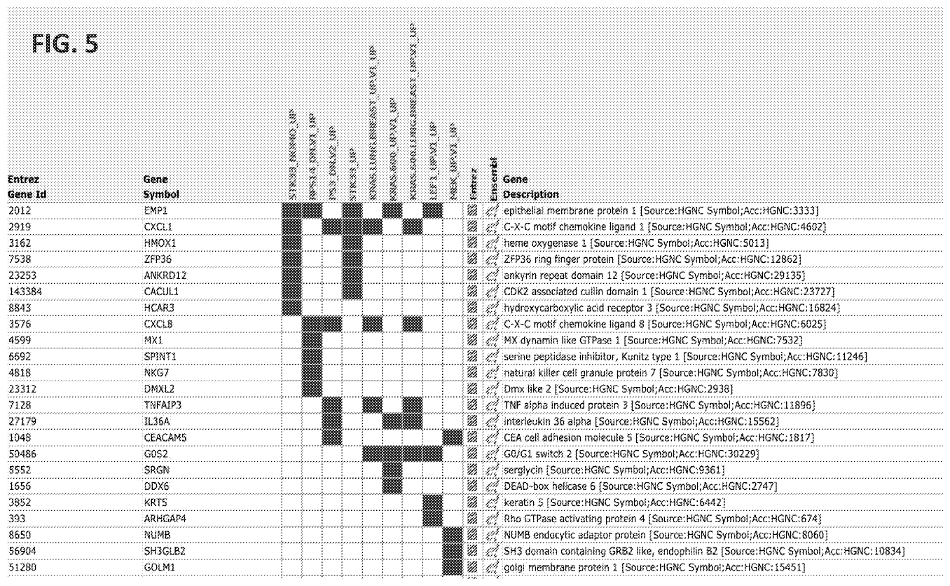

28. The method of embodiment 1, wherein the feature set used by the classification algorithm includes one or more features selected from a geneset of any of FIGS. 2, 3, 4 and 5.

29. The method of embodiment 1, wherein the feature set used by the classification algorithm includes an activity of microbial taxon or one or more taxa of FIG. 6, e.g., Streptococcus, Rothia, Eikenella, Abiotrophia, Fusobacterium, Selenomonas, Capnocytophaga, Prevotella, Actinomyces, or Veillonella.

30. The method of embodiment 1, wherein the feature set used by the classification algorithm includes an activity of one or more microbial gene orthologs of FIG. 7A-7B, e.g., opportunistic microbial activities, oral pathobionts, LPS production, biofilm and virulence pathways, hydrogen sulfide production, alternative sugar metabolism and energy utilization, glutathione production and transport, nitrate reduction, ammonia production and lysine, cadaverine and putrescine production.

31. The method of embodiment 1, wherein the cancer is oral squamous cell carcinoma (“OSCC”).

32. The method of embodiment 31, wherein the inference is likely presence of OSCC″ or “unlikely presence of OSCC.”

33. The method of embodiment 1, wherein the oral cancer is selected from squamous cell carcinoma, verrucous carcinoma, minor salivary gland carcinoma, lymphoma, benign oral cavity tumor and basal cell carcinoma.

34. The method of embodiment 1, wherein the classification model classifies presence or absence of oral cancer.

35. The method of embodiment 1, wherein the classification model classifies a stage of oral cancer (e.g., selected from stage 0, stage 1, stage 2, stage 3, stage 4).

36. The method of embodiment 1, wherein the classification model is selected to have a sensitivity of at least 90% and a selectivity of at least 90%.

37. The method of embodiment 1, further comprising:

e) outputting the inference to a user interface device or to computer-readable memory.

38. The method of embodiment 1, further comprising:

e) delivering and/or administering to the subject a therapeutic intervention effective to treat the oral cancer.

39. The method of embodiment 1, further comprising:

e) for a subject inferred to have oral cancer, performing a confirmatory diagnostic step selected from biopsy or imaging.

40. A method comprising:

a) providing biological samples from each of a first set of subjects and a second set of subjects, wherein the biological samples comprise an oral microbiome, and, optionally, somatic host cells, and wherein the first set of subjects have oral cancer present and the second set of subjects have oral cancer absent;
b) sequencing nucleic acids in the biological samples to provide sequence information; and
c) performing a statistical analysis on the sequence information to produce a model that infers a state of oral cancer in a subject based on sequence information.

41. The method of embodiment 40, wherein the statistical analysis comprises a model developed by machine learning.

42. The method of embodiment 40, wherein the statistical analysis comprises an analysis selected from correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) and non-parametric analysis (e.g., Wilcoxon rank-sum test, Wilcoxon sign-rank test, sign test).

43. A method comprising:

a) administering to a subject inferred to have oral cancer by a method of embodiment 1, a therapeutic intervention effective to treat the oral cancer.

44. The method of embodiment 43, wherein the therapeutic intervention is selected from surgical removal of cancerous tissue; administration of a chemotherapeutic agent; and administration of a dietary supplement, a food ingredient, or a food that diminishes a dysbiosis in oral microbiome of the subject associated with the cancer.

45. The method of embodiment 43, wherein the therapeutic intervention comprises one or more of:

1) increasing the abundance of an under-represented taxon;
2) reducing the abundance of an over-represented taxon;
3) reducing the abundance of a microbial function;
4) increasing the abundance of a microbial function;
5) decreasing interactions between microorganisms or their molecules (metabolites, nucleic acids, proteins) and human tissue that support cancer onset or progression; and
6) enhancing the interactions between microorganisms or their molecules (metabolites, nucleic acids, proteins) and human tissue that inhibit cancer onset or progression.

46. A system comprising:

(a) a computer comprising: (i) a processor; and (II) a memory, coupled to the processor, the memory storing a module comprising:
(1) nucleic acid sequence information from a biological sample from a subject comprising an oral microbiome;
(2) a classification model which, based on values including the measurements, classifies the subject as having oral cancer present or absent, wherein the classification model is selected to have a sensitivity of at least 75%, at least 85% or at least 95%; and
(3) computer executable instructions for implementing the classification model on the test data.

47. A method for developing a computer model for inferring, from feature data, a state of oral cancer in a subject, the method comprising:

a) training a machine learning algorithm on a training data set, wherein the training data set comprises, for each of a plurality of subjects, (1) a class label classifying a subject as having or not having an oral cancer; and (2) feature data comprising quantitative measures for each of a plurality of features selected from oral microbiome transcriptome expression, and wherein the machine learning algorithm develops a model that infers a class label for a subject based on the feature data.

48. A method that infers a state of oral cancer in a subject, the method comprising:

(a) providing a data set comprising, for the subject, feature data for each of a plurality of features selected from oral microbiome transcriptome gene expression data and taxa activity data; and
(b) executing a computer model on the data set to infer the presence or absence of oral cancer in the subject.

49. A software product comprising a computer readable medium in tangible form comprising machine executable code, which, when executed by a computer processor, infers a state of oral cancer in a subject by:

(a) accessing a data set comprising, for a subject, feature data for each of a plurality of features selected from oral microbiome transcriptome gene expression data and taxa activity data; and
(b) executing a computer model on the data set to infer the state of oral cancer in the subject.

50. A method of treating oral cancer in a subject comprising:

(a) inferring the presence of oral cancer in a subject according to a method as described herein; and
(b) administering a therapeutic intervention to the subject effective to treat the oral cancer.

51. A method for diagnosing and treating an oral cancer in a subject, the method comprising:

(a) receiving from a subject a sample comprising an oral microbiome and, optionally, host somatic cells;
(b) determining nucleic acid sequences of a microorganism component of the sample;
(c) determining alignments of the nucleic acid sequence to reference nucleic acid sequences associated with the oral cancer;
(d) generating a microbiome feature dataset for the subject based upon the alignments;
(e) generating an inference of the oral cancer in the subject upon processing the microbiome feature dataset with an inference model derived from a population of subjects; and
(f) at an output device associated with the subject, providing a therapy to the subject with the oral cancer upon processing the inference with a therapy model designed to treat the oral cancer.

52. A method comprising:

(a) measuring, in a sample from a subject comprising an oral microbiome and, optionally, host somatic cells, activity of one or more biomarkers selected from Table 1, Table 2, Table 3 and/or Table 4;
(b) inferring, from the measurements, presence of oral cancer in the subject; and
(c) delivering to the subject a therapeutic intervention to treat the oral cancer.

53. The method of embodiment 52, wherein measuring comprises:

(i) optionally, amplifying microbial metatranscriptome sequences in the sample;
(ii) sequencing the microbial metatranscriptome from the sample to produce sequence reads;
(iii) searching reference sequences in a reference sequence catalog for matches with the sequence reads;
(iv) determining amounts of sequence reads matching references sequences in the catalog to produce a data set; and
(v) determining, from the data set, activity of each of the one or more biomarkers.

54. The method of embodiment 53, wherein determining activity comprises:

(1) for biomarkers that are taxa categories, performing a taxonomic analysis with a metagenomic classifier to measure taxa activity;
(2) for biomarkers that are gene orthologs, performing a functional analysis by determining activity of genes having the same function across taxa based on sequences corresponding to microbial open reading frames (ORFs), and combing the activities to produce gene ortholog activity.

55. The method of embodiment 52, wherein inferring comprises:

(i) executing by computer a classification model that infers presence or absence of oral cancer based on the biomarkers.

56. The method of embodiment 52, wherein measuring comprises:

(i) selectively amplifying in the sample nucleic acids specific for the biomarkers; and
(ii) determining amounts of the amplified nucleic acids.

57. A method comprising:

a) providing biological samples from each of a first set of subjects and a second set of subjects having an oral cancer and having been subject to a therapeutic intervention, wherein the biological samples comprise an oral microbiome, and, optionally, host somatic cells, and wherein the first set of subjects responded positively to the therapeutic intervention and the second set of subjects did not respond positively to the therapeutic intervention;
b) sequencing nucleic acids in the biological samples to provide sequence information; and
c) performing a statistical analysis on the sequence information to produce a model that infers subject oral cancer having a positive response or lack of positive response to the therapeutic intervention.

58. A method of treating a subject with oral cancer comprising:

(a) inferring that the subject will respond positively to each of one or more therapeutic interventions by executing a model on nucleic acid information from a biological sample from the subject comprising or oral microbiome and, optionally, host somatic cells; and
(b) administering to the subject one or more therapeutic interventions to treat the cancer.

59. A method comprising:

(a) identifying a subject inferred to have oral cancer by a method of embodiment 1; and
(b) performing imaging or biopsy to confirm the inference.

60. The method of embodiment 59, wherein the oral cancer is squamous cell carcinoma (“OSCC”).

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A method comprising:
a) providing a biological sample from a subject comprising mouth-sourced cells;
b) sequencing nucleic acids from the sample to produce sequence information;
c) determining, from the sequence information, (1) measures of activity of one or more microbial taxa, (2) measures of activity of one or more microbial gene orthologs, and/or (3) measures of activity of one or more somatic cell genes of the subject, wherein the one or more measures are included in a feature set;
d) executing by computer a classification model that infers, from one or more features in the feature set, a state of oral cancer in the subject.

2. The method of claim 1, wherein the biological sample comprises saliva.

3. The method of claim 1, wherein the biological sample comprises microbial cells and host cells.

4. The method of claim 1, wherein the subject is a human.

5. The method of claim 1, wherein the subject is over 50 years of age or has a history of tobacco use.

6. The method of claim 1, wherein the mouth-sourced cells comprise an oral microbio and, optionally, somatic cells from the subject.

7. The method of claim 6, wherein the somatic cells from the subject comprise cells selected from cheek cells, gum cells and tongue cells.

8. The method of claim 1, wherein the nucleic acids sequenced comprise mRNA and the sequence information comprises metatranscriptomic information.

9. The method of claim 1, wherein the feature set used by the classification algorithm includes at least: (1) measures of activity of one or more microbial taxa.

10. The method of claim 9, wherein the feature set used by the classification algorithm further includes: (2) measures of activity of one or more microbial gene orthologs.

11. The method of claim 10, wherein the feature set used by the classification algorithm further includes: (3) measures of activity of one or more host somatic cell genes.

12. The method of claim 1, wherein the feature set used by the classification algorithm includes at least two of: (1) measures of activity of one or more microbial taxa, (2) measures of activity of one or more microbial gene orthologs, or (3) measures of activity of one or more somatic cell genes of the subject.

13. The method of claim 1, wherein the classification model uses one or more features selected from the features of Table 1.

14. The method of claim 1, wherein the classification model uses at least, exactly or no more than any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, or 157 of the features selected from the features of Table 1.

15. The method of claim 1, wherein the classification model uses at least, exactly or no more than any of 1, 2, 3, 4, 5, 6, 7, 8, 9, 10, 11, 12, 13, 14, 15, 16, or 17 of the features selected from: Actinobaculum sp. oral taxon 183, Actinomyces massiliensis, Actinomyces sp. oral taxon 448, Alloscardovia omnicolens, Selenomonas sp. CM52, Mycoplasma salivarium, Parvimonas sp. oral taxon 110, Rothia sp. HMSC062H08, K01697, K12452, Actinomyces johnsonii, Prevotella loescheii, Streptococcus cristatus, Streptococcus sobrinus, Streptococcus sp. HPH0090, Tannerella forsythia, and K02909.

16. The method of claim 15, wherein the features of Table 1 include one or more microbial taxa features and/or one or more gene ortholog features.

17. The method of claim 15, wherein the features of Table 1 include one or more positively associated features and/or one or more negatively associated features.

18. The method of claim 1, wherein the classification model uses only features selected from the features of Table 1.

19. The method of claim 1, wherein the feature set used by the classification algorithm includes at least 30, at least 50, at least 100, at least 200 or all of the features selected from Tables 2, 3 or 4.

20. The method of claim 19, wherein the feature set used by the classification algorithm includes at least 10 microbial taxa features, at least 10 microbial gene ortholog features and at least 10 host cell gene features.

21. The method of claim 19, wherein the feature set used by the classification algorithm further includes: mechanism feature, a toxic burden feature (3) measures of activity of one or more host somatic cell genes.

22. The method of claim 19, wherein the features of Table 1 include one or more microbial taxa features and/or one or more gene ortholog features.

23. The method of claim 19, wherein the features of Table 1 include one or more positively associated features and/or one or more negatively associated features.

24. The method of claim 1, wherein the classification model uses only features selected from the features of Tables 2, 3 and 4.

25. The method of claim 1, wherein the classification model uses at least, exactly or no more than any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, or 270 of the features selected from the features of Tables 2, 3 or 4.

26. The method of claim 1, wherein the feature set used by the classification algorithm includes one or more features selected from a pro-inflammatory activity feature, a hydrogen sulfide production activity feature, a microbial contribution to cancer-specific energy metabolism feature, a protein fermentation as a tumor genic mechanism feature, tox burden feature, and microbial antibiotic resistance in tumorigenesis feature.

27. The method of claim 26, wherein the selected features are from Table 5.

28. The method of claim 1, wherein the feature set used by the classification algorithm includes one or more features selected from a geneset of any of FIGS. 2, 3, 4 and 5.

29. The method of claim 1, wherein the feature set used by the classification algorithm includes an activity of microbial taxon or one or more taxa of FIG. 6, e.g., Streptococcus, Rothia, Eikenella, Abiotrophia, Fusobacterium, Selenomonas, Capnocytophaga, Prevotella, Actinomyces, or Veillonella.

30. The method of claim 1, wherein the feature set used by the classification algorithm includes an activity of one or more microbial gene orthologs of FIG. 7A-7B, e.g., opportunistic microbial activities, oral pathobionts, LPS production, biofilm and virulence pathways, hydrogen sulfide production, alternative sugar metabolism and energy utilization, glutathione production and transport, nitrate reduction, ammonia production and lysine, cadaverine and putrescine production.

31. The method of claim 1, wherein the cancer is oral squamous cell carcinoma (“OSCC”).

32. The method of claim 31, wherein the inference is likely presence of OSCC” or “unlikely presence of OSCC.”

33. The method of claim 1, wherein the oral cancer is selected from squamous cell carcinoma, verrucous carcinoma, minor salivary gland carcinoma, lymphoma, benign oral cavity tumor and basal cell carcinoma.

34. The method of claim 1, wherein the classification model classifies presence or absence of oral cancer.

35. The method of claim 1, wherein the classification model classifies a stage of oral cancer (e.g., selected from stage 0, stage 1, stage 2, stage 3, stage 4).

36. The method of claim 1, wherein the classification model is selected to have a sensitivity of at least 90% and a selectivity of at least 90%.

37. The method of claim 1, further comprising:
e) outputting the inference to a user interface device or to computer-readable memory.

38. The method of claim 1, further comprising:
e) delivering and/or administering to the subject a therapeutic intervention effective to treat the oral cancer.

39. The method of claim 1, further comprising:
e) for a subject inferred to have oral cancer, performing a confirmatory diagnostic step selected from biopsy or imaging.

40. A method comprising:
a) providing biological samples from each of a first set of subjects and a second set of subjects, wherein the biological samples comprise an oral microbiome, and, optionally, somatic host cells, and wherein the first set of subjects have oral cancer present and the second set of subjects have oral cancer absent;
b) sequencing nucleic acids in the biological samples to provide sequence information; and
c) performing a statistical analysis on the sequence information to produce a model that infers a state of oral cancer in a subject based on sequence information.

41. The method of claim 40, wherein the statistical analysis comprises a model developed by machine learning.

42. The method of claim 40, wherein the statistical analysis comprises an analysis selected from correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) and non-parametric analysis (e.g., Wilcoxon rank-sum test, Wilcoxon sign-rank test, sign test).

43. A method comprising:
a) administering to a subject inferred to have oral cancer by a method of claim 1, a therapeutic intervention effective to treat the oral cancer.

44. The method of claim 43, wherein the therapeutic intervention is selected from surgical removal of cancerous tissue; administration of a chemotherapeutic agent; and administration of a dietary supplement, a food ingredient, or a food that diminishes a dysbiosis in oral microbiome of the subject associated with the cancer.

45. The method of claim 43, wherein the therapeutic intervention comprises one or more of:
1. increasing the abundance of an under-represented taxon;
2. reducing the abundance of an over-represented taxon;
3. reducing the abundance of a microbial function;
4. increasing the abundance of a microbial function;
5. decreasing interactions between microorganisms or their molecules (metabolites, nucleic acids, proteins) and human tissue that support cancer onset or progression; and
6. enhancing the interactions between microorganisms or their molecules (metabolites, nucleic acids, proteins) and human tissue that inhibit cancer onset or progression.

46. A system comprising:
(a) a computer comprising: (i) a processor; and (II) a memory, coupled to the processor, the memory storing a module comprising:
(1) nucleic acid sequence information from a biological sample from a subject comprising an oral microbiome;
(2) a classification model which, based on values including the measurements, classifies the subject as having oral cancer present or absent, wherein the classification model is selected to have a sensitivity of at least 75%, at least 85% or at least 95%; and
(3) computer executable instructions for implementing the classification model on the test data.

47. A method for developing a computer model for inferring, from feature data, a state of oral cancer in a subject, the method comprising:
a) training a machine learning algorithm on a training data set,
wherein the training data set comprises, for each of a plurality of subjects, (1) a class label classifying a subject as having or not having an oral cancer; and (2) feature data comprising quantitative measures for each of a plurality of features selected from oral microbiome transcriptome expression, and
wherein the machine learning algorithm develops a model that infers a class label for a subject based on the feature data.

48. A method that infers a state of oral cancer in a subject, the method comprising:
(a) providing a data set comprising, for the subject, feature data for each of a plurality of features selected from oral microbiome transcriptome gene expression data and taxa activity data; and
(b) executing a computer model on the data set to infer the presence or absence of oral cancer in the subject.

49. A software product comprising a computer readable medium in tangible form comprising machine executable code, which, when executed by a computer processor, infers a state of oral cancer in a subject by:
(a) accessing a data set comprising, for a subject, feature data for each of a plurality of features selected from oral microbiome transcriptome gene expression data and taxa activity data; and
(b) executing a computer model on the data set to infer the state of oral cancer in the subject.

50. A method of treating oral cancer in a subject comprising:
(a) inferring the presence of oral cancer in a subject according to a method as described herein; and
(b) administering a therapeutic intervention to the subject effective to treat the oral cancer.

51. A method for diagnosing and treating an oral cancer in a subject, the method comprising:
(a) receiving from a subject a sample comprising an oral microbiome and, optionally, host somatic cells;
(b) determining nucleic acid sequences of a microorganism component of the sample;
(c) determining alignments of the nucleic acid sequence to reference nucleic acid sequences associated with the oral cancer;
(d) generating a microbiome feature dataset for the subject based upon the alignments;
(e) generating an inference of the oral cancer in the subject upon processing the microbiome feature dataset with an inference model derived from a population of subjects; and
(f) at an output device associated with the subject, providing a therapy to the subject with the oral cancer upon processing the inference with a therapy model designed to treat the oral cancer.

52. A method comprising:
(a) measuring, in a sample from a subject comprising an oral microbiome and, optionally, host somatic cells, activity of one or more biomarkers selected from Table 1, Table 2, Table 3 and/or Table 4;
(b) inferring, from the measurements, presence of oral cancer in the subject; and
(c) delivering to the subject a therapeutic intervention to treat the oral cancer.

53. The method of claim 52, wherein measuring comprises:
(i) optionally, amplifying microbial metatranscriptome sequences in the sample;
(ii) sequencing the microbial metatranscriptome from the sample to produce sequence reads;
(iii) searching reference sequences in a reference sequence catalog for matches with the sequence reads;
(iv) determining amounts of sequence reads matching references sequences in the catalog to produce a data set; and
(v) determining, from the data set, activity of each of the one or more biomarkers.

54. The method of claim 53, wherein determining activity comprises:
(1) for biomarkers that are taxa categories, performing a taxonomic analysis with a metagenomic classifier to measure taxa activity;
(2) for biomarkers that are gene orthologs, performing a functional analysis by determining activity of genes having the same function across taxa based on sequences corresponding to microbial open reading frames (ORFs), and combing the activities to produce gene ortholog activity.

55. The method of claim 52, wherein inferring comprises:
(i) executing by computer a classification model that infers presence or absence of oral cancer based on the biomarkers.

56. The method of claim 52, wherein measuring comprises:
(i) selectively amplifying in the sample nucleic acids specific for the biomarkers; and
(ii) determining amounts of the amplified nucleic acids.

57. A method comprising:
a) providing biological samples from each of a first set of subjects and a second set of subjects having an oral cancer and having been subject to a therapeutic intervention, wherein the biological samples comprise an oral microbiome, and, optionally, host somatic cells, and wherein the first set of subjects responded positively to the therapeutic intervention and the second set of subjects did not respond positively to the therapeutic intervention;
b) sequencing nucleic acids in the biological samples to provide sequence information; and
c) performing a statistical analysis on the sequence information to produce a model that infers subject oral cancer having a positive response or lack of positive response to the therapeutic intervention.

58. A method of treating a subject with oral cancer comprising:
(a) inferring that the subject will respond positively to each of one or more therapeutic interventions by executing a model on nucleic acid information from a biological sample from the subject comprising or oral microbiome and, optionally, host somatic cells; and
(b) administering to the subject one or more therapeutic interventions to treat the cancer.

59. A method comprising:
(a) identifying a subject inferred to have oral cancer by a method of claim 1; and
(b) performing imaging or biopsy to confirm the inference.

60. The method of claim 59, wherein the oral cancer is squamous cell carcinoma (“OSCC”).
